(19) United States
(12) Patent Application Publication
(10) Pub. No.: US 2009/0318746 A1
(43) Pub. Date: Dec. 24, 2009

(54) Lubricious echogenic coatings

(75) Inventors: Kenneth Bruce Thurmond, II, Pitt Meadows (CA); Robert Lyon Henn, Raleigh, NC (US); Rui Avelar, Vancouver (CA)

Correspondence Address: STEPTOE & JOHNSON LLP, 1330 CONNECTICUT AVENUE, N.W., WASHINGTON, DC, 20036, US

(73) Assignee: ANGIOTECH BIOCOATINGS CORP., Henrietta, NY (US)

(21) Appl. No.: 12/162,560

(22) PCT Filed: Jan. 31, 2007
(86) PCT No.: PCT/US07/02504

§ 371 Date: May 20, 2009

Related U.S. Application Data
(60) Provisional application No. 60/763,361, filed on Jan. 31, 2006.
(60) Provisional application No. 60/763,920, filed on Feb. 1, 2006.
(60) Provisional application No. 60/835,086, filed on Aug. 3, 2006.

(51) Int. Cl. A61L 29/16; A61L 31/08; A61L 27/28; A61M 36/12; A61M 29/02; B05D 3/00; A61K 49/22

(52) U.S. Cl. 600/8; 604/265; 604/246; 623/1.46; 606/191; 606/200; 427/2.1; 427/2.25; 424/9.5

(57) ABSTRACT

Medical devices can include a lubricious echogenic coating that is slippery when wet and provides visibility during ultrasonic imaging.

### CLAIM OF PRIORITY

This application claims priority to provisional U.S. Patent Application No. 60/763,361, titled “Lubricious Coating for Surgical Instruments,” filed Jan. 31, 2006, to provisional U.S. Patent Application No. 60/763,920, titled “Lubricious Echogenic Coating for Surgical Instruments,” filed Feb. 1, 2006, and to provisional U.S. Patent Application No. 60/835,086, titled “Lubricious Coating for Surgical Instruments,” filed Aug. 3, 2006, each of which is incorporated by reference in its entirety.

### TECHNICAL FIELD

The invention relates to lubricious echogenic coatings.

### BACKGROUND

A health care provider seeks to carry out operations with a high degree of accuracy and precision so as to minimize unnecessary trauma and/or damage to a patient. The health care provider uses a variety of instruments to perform surgical procedures, including, for example, dissection, curetting, suturing, and cutting with scissors or a scalpel, and many others known in the art. Because the instrument contacts the patient, the properties of the instrument can influence the extent to which a patient's tissues suffer undesired trauma or damage. The health care provider also contacts the instrument, and so the properties of the instrument can influence how effectively the health care provider can use the instrument.

In addition, the ability to accurately place a device within a tissue or passageway, e.g., intravascularly, can be very important to complete the diagnosis or therapy of a patient. Hydrophilic, lubricant and/or echogenic coatings can make medical devices slippery when wet and/or highly echogenic and readily recognizable from surrounding tissue or fluid under ultrasound imaging.

### SUMMARY

Medical devices can be coated with hydrophilic, lubricant, and optionally echogenic coatings. Such coated devices can thus be slippery when wet, yet non-slippery when dry, and optionally echogenic. The coated devices can be provided in a dry state, so as to be non-slippery for ease of handling and preparation. Once the device is wetted, before or during a medical procedure, it can become slippery so as to protect the patient and can reduce friction and damage to surrounding tissue. A device that has an echogenic coating can be readily distinguished from surrounding tissue or fluid when observed by ultrasound imaging.

Medical devices may include appropriate dye components to make them optically visible during surgical procedures. The coating can be employed to reduce the coefficient of friction of instruments including, for example, knives, scalpels, rongeurs, dissectors, scissors, needle drivers, suture holders, curettes, electrodes, probes, forceps, aneurysm clip applicators, and the like. The coatings can enhance the ultrasound visibility of surfaces of needles, catheters, and laparoscopic devices, such as intravascular retrieval snares or baskets.

In one aspect, a medical device includes a surface coated with a first layer proximal to the surface, a second layer coated on the first layer, the second layer including a plurality of microparticles, and a third layer coated on the second layer, at least one layer including an echogenic structure.

The first layer can include an ethylene acrylic acid copolymer and an isocyanate. The second layer can include a plurality of microparticles. The plurality of microparticles can include a polymeric gas/liquid containing microparticle. The third layer can include a cellulose based polymer.

In another aspect, a medical device includes a surface coated with a first layer proximal to the surface, the first layer including an ethylene acrylic acid copolymer and an isocyanate; a second layer coated on the first layer, the second layer including a plurality of microparticles; and a third layer coated on the second layer, the third layer including a cellulose based polymer.

The isocyanate of the first layer can be an aromatic isocyanate. The first layer can further include an acrylic polymer and a melamine formaldehyde polymer. The plurality of microparticles can include a polymeric gas/liquid containing microparticle. The third layer can further include a polyurethane. The third layer can further include a polyvinylpyrrolidone.

The device can be a needle, a vascular dilator, a guide wire, a stent, a biopsy site marker, a retrieval snare, a trocar, a hemostasis valve, or a brachytherapy seed. The needle can be a disposable soft tissue biopsy needle, an aspiration needle, a bone marrow biopsy needle, a breast localization needle, an injection needle, a biopsy co-axial introducer needle, a bone biopsy needle, a guidewire introducer needle, an epidural needle, or a Huber needle. The device can be a vascular retrieval snare.

In another aspect, a method of coating a surface of a device includes forming a coating on the surface, wherein the coating includes an echogenic structure and a plurality of microparticles. Forming the coating can include contacting the surface with a first coating solution including a first solvent, an ethylene acrylic acid copolymer and an isocyanate. Forming the coating can include contacting the surface with a second coating solution including a second solvent and a plurality of microparticles. Forming the coating can include contacting the surface with a third coating solution including a third solvent and a cellulose based polymer.

The isocyanate of the first coating solution can be an aliphatic or aromatic isocyanate. The first coating solution can further include an acrylic polymer and a melamine formaldehyde polymer. The plurality of microparticles can include a polymeric gas/liquid containing microparticle. The third coating solution can further include a polyurethane. The third coating solution can further include a polyvinylpyrrolidone.

The device can be a needle, a vascular dilator, a guide wire, a stent, a biopsy site marker, a retrieval snare, a trocar, a hemostasis valve, or a brachytherapy seed. The needle can be a disposable soft tissue biopsy needle, an aspiration needle, a bone marrow biopsy needle, a breast localization needle, an injection needle, a biopsy co-axial introducer needle, a bone biopsy needle, a guidewire introducer needle, an epidural needle, or a Huber needle. The device can be a vascular retrieval snare.

In another aspect, a medical device can include a coating on a surface of the device, wherein the coating comprises a plurality of echogenic microparticles. The plurality of microparticles can include a polymeric microparticle containing a gas or liquid. The polymeric microparticle can include isopentane, isobutane, a fluorocarbon, air, nitrogen, carbon dioxide, argon, helium, or oxygen.

The coating can include a first layer proximal to the surface of the device. The coating further can include a second layer coated on the first layer. The coating further can include a third layer coated on the second layer.

The surface can be stainless steel, a stainless steel alloy, nitinol, titanium, a titanium alloy, tantalum, aluminum, a cobalt-chromium alloy, a nickel-silver alloy, polyethylene, polypropylene, a fluoropolymer, a polyvinyl chloride, polyethylene terephthalate, polymethyl methacrylate, or silicone.

The device can be a needle, a vascular dilator, a guide wire, a stent, a biopsy site marker, a retrieval snare, a trocar, a hemostasis valve, catheters, or a brachytherapy seed. The catheter can be a drainage catheter. The device can be a vascular access needle. The device can be a vascular retrieval snare.

The needle can be a soft tissue biopsy needle, an aspiration needle, a bone marrow biopsy needle, a breast localization needle, an injection needle, a biopsy co-axial introducer needle, a bone biopsy needle, a guidewire introducer needle, an epidural needle, or a Huber needle. The needle can be disposable.

The first layer can include an isocyanate. The isocyanate can be an aromatic isocyanate. The first layer can include an acrylic polymer and a melamine formaldehyde polymer. The first layer can include a vinyl acetate-acrylic copolymer and a formaldehyde copolymer. The first layer can include an ethylene acrylic acid copolymer and an epoxy-bisphenol A copolymer. The first layer further can include a polyurethane. The polyurethane can be an aliphatic polycarbonate based polyurethane.

The second layer can include a cellulose based polymer. The second layer further can include a polyurethane. The second layer further can include a polyvinylpyrrolidone. The second layer further can include a silicon based polymer.

The third layer can include a cellulose based polymer. The third layer further can include a polyurethane. The third layer further can include a polyvinylpyrrolidone. The third layer further can include a silicone.

The microparticles can be substantially localized in the second layer.

The coating can be a lubricious coating.

The first layer can include the microparticles and the second layer can be lubricious.

In another aspect, a composition includes a plurality of echogenic microparticles suspended in a first polymer matrix. The first polymer matrix can include an ethylene acrylic acid copolymer, an isocyanate, a cellulose based polymer, an acrylic polymer, a melamine formaldehyde polymer, a polyurethane, or a polyvinylpyrrolidone. The plurality of microparticles can include a polymeric gas/liquid containing microparticle.

The composition can further include a solvent. The first polymer matrix can be coated on a second polymer matrix.

Advantageous properties of the coating modified medical devices allows surgeons to maintain very fine control of surgical procedures, so as to minimize the invasiveness of such surgical procedures, and thereby to enhance patient recovery and surgical outcome.

### BRIEF DESCRIPTION OF THE DRAWINGS

FIG. 1 is a depiction of an intravascular retrieval snare.

### DETAILED DESCRIPTION

A medical device can have a coating on an external surface. The coating can be abrasion resistant, lubricious, echogenic and biocompatible. A portion of, or the entire surface of, the medical device may be provided with a coating. In some cases, a portion of the device (such as, for example, a handle) is uncoated so that the uncoated portion provides a higher friction surface than a coated portion. In some cases, an inner portion (such as a part or all of a lumen) may be provided with a coating.

Coatings may be continuous or discontinuous (e.g., patterned or covering only portions of the surface) and may be of uniform thickness or may be of uneven thickness. Coatings may be deposited into divots, voids, or grooves in the structure of the device to provide discrete deposits of material. The coating can be applied selectively to a surface of a device, such that desired portions of the surface are coated while other portions remain uncoated. The coating can be discontinuous, i.e., there can be local regions that lack coating, whether the discontinuous nature is desired or unintentional.

The medical device (or at least a portion of it) is to be coated with at least one layer, providing the top coat or external layer. The external layer can be directly adhered to a surface of the device. Alternatively, one or more intermediate layers can be present between the surface of the device and the external layer.

The medical devices can be coated by applying a coating material to the surface of the medical device. For example, the coating material can be dissolved in a solvent, the resulting solution contacted to the device, and the solvent removed. The coating may be applied using standard coating methods, such as by spraying, dipping, roll coating, bar coating, spin coating, or wiping, or may be manufactured using an extrusion process. The coating may be applied as a solution, and then the solvent allowed to evaporate. Evaporation can be promoted by an elevated temperature. In some cases, kits include a medical device, coated or uncoated and are provided with a swab, which can be wetted with a coating material for coating the surface of the instrument. The kit can be useful in circumstances where it is desirable to apply the coating to the device a short time before the device is used in a medical procedure.

When the coating includes more than one layer (i.e., at least one intermediate layer in addition to the external layer), the layers can be sequentially applied to the device to form the coating. For example, if the coating includes one intermediate layer and an external layer, the intermediate layer can be applied to the device and dried; the external layer is then subsequently applied. The surface of the device can be substantially inert. For example, the surface can be substantially free of reactive functional groups. In some cases, “substantially free of reactive functional groups” simply means that the surface is used as-prepared, with such reactive functional groups as may normally be present in a surface of that particular material, and no exogenous reactive functional groups are added to the surface.

An intermediate layer can be a bonding layer selected to promote adhesion of the external layer to the device. The bonding layer can promote abrasion resistance of the outer layer, and prolong adhesion of the outer layer to the after soaking in water, when compared to a coating without the bond coat layer. The coating can remain adhered to the device when subjected to bending through a small radius. Depending on the substrate material, an additional primer (pre-coat) layer may be used to further improve the adhesion of the bonding and/or lubricious coating layers to the substrate.

Any layer within the coating system can include microparticles. Microparticles can be formed by a variety of methods, including spray-drying methods, milling methods, coacervation methods, W/O emulsion methods, W/O/W emulsion methods, and solvent evaporation methods. These techniques allow for the formation of microparticles that are solid, hollow, liquid filled, or gas filled and have the capability to be formed using any number of polymers or polymer combinations. Representative examples of polymers that may be used to form microparticles include polyvinylidene chloride, polyacrylonitrile, polymethylmethacrylate, poly(acrylates), poly(methacrylates), crosslinked acrylic or methacrylic polymers, polyurethanes, polyesters, poly(vinyl ethers), poly(amides), polycarbonates, polystyrene, poly(isobutylene), poly(acrylic acid), poly(vinyl acetate), epoxy resins, phenolic resins, nylon, poly(lactic acid), poly(glycolic acid) and copolymers and/or combinations thereof.

Microparticles may take shape, although the particles frequently are spherical in shape. Diameters of microparticles may range in size from about 100 nm to 1000 μm. Microparticles with dimensions less than 1 micron also may be referred to as nanoparticles. Typically, microparticles having diameters from about 0.1 μm to about 30 μm can be generally preferred for use in echogenic coatings. The useful particle size depends on the coating thickness, as there has to be enough coating present to at least partially include the microparticle in order to maintain adhesion between the microparticle and the coating. Additionally, the concentration of the microparticles within the polymer coating is important as it will balance the echogenicity with the smoothness of the coating surface. Microparticle concentrations from 0.01% to 50% by weight are possible, with the preferred concentrations being about 0.1% to about 10%.

Including microparticles in one of the coating layers can produce a medical device which, surprisingly, can remain visible under ultrasound in vivo an extended period of time (e.g., weeks or months), significantly longer than devices with coatings that lack microparticles. The microparticles can be, for example, polymeric gas or liquid (e.g., isopentane or isobutane, which boil at 28° C. and −12° C., respectively) containing microparticles (e.g., EXPANCEL microparticles), air filled galactose microparticles (e.g., ECHOVIST or LEVOVIST), fluorocarbon filled degradable poly(glycoliclactic acid) microparticles (AI-100 by Acusphere), as well as others known to those skilled in the art. Additionally, inclusion of the microparticles into any layer allows for the tailoring of the top layer depending upon the ultimate use of the device. Use of echogenic microparticles into one or more coating layers allows for much flexibility in the type and properties of the coatings that may be used. For example, when microparticles are incorporated into a coating base layer, the device may be further coated with a lubricous top layer to provide a device that is both echogenic and lubricious when wet. Alternatively, or in addition, particles may be incorporated into compositions to provide a tough or hard coating. Tough echogenic coatings may be particularly useful in applications requiring a coating that will not be altered or destroyed during use.

Examples of solvents useful for applying the coatings to a device can include butyrolactone, alcohols (e.g., methanol, ethanol, isopropanol, n-butyl alcohol, i-butyl alcohol, t-butyl alcohol, and the like), dimethyl acetamide, and n-methyl-2-pyrrolidone. Many other types of solvents may be of use in applying coatings, as well. These solvents and others cause different degrees of swelling of a plastic substrate or inner layer, as the case may be. The duration and temperature of solvent evaporation may be selected to achieve stability of the coating layer and to achieve a bond between the surface being coated and the coating layer. It is possible to control the degree of stability, wet lubricity, insolubility, flexibility, and adhesion of the coating by varying the weight-to-volume percentages of the components in the coating solutions.

The coating can be dried at temperatures between 50° C. and 120° C., but may be done at higher or lower temperatures. Advantageously, hydrophilic coatings can cure faster than a silicone based coating. After drying, the top coat polymer layer is left partially embedded in a polymer surface and/or partially in the case of the two-layer system; the solvent used during the coating application can be too active such that the top coat penetrates into the polymer surface to such a degree that the coated layer behaves as though it has been highly cross-linked. This causes the top coat to become not sufficiently swollen and lubricious when wet by aqueous fluids. Solvent mixtures can also be too inactive, so that the coating is not resistant enough to abrasion when wet and is too easily removed. Other polymers or cross-linking agents may be incorporated with the hydrophilic polymer(s) in the lubricious layer to enhance the adhesion of the layer to the polymer surface, making the lubricious layer more resistant to wet abrasion.

The external layer can include a hydrophilic polymer. The hydrophilic polymer of the external layer can include poly(vinylpyrrolidone) (PVP) or a PVP-vinylacetate copolymer. The hydrophilic polymer of the external layer can have a molecular weight of, for example, greater than 100,000, greater than 150,000, greater than 200,000, greater than 250,000, greater than 300,000, greater than 350,000, or greater than 400,000. In some cases, the hydrophilic polymer of the external layer has a molecular weight in the range of 120,000-360,000. An intermediate layer can include PVP of lower molecular weight, e.g., as low as 15,000. A PVP-vinylacetate copolymer can be used in place of PVP.

The external layer can include a stabilizing polymer. A stabilizing polymer may serve as a component in a coating layer to help bind the lubricious polymer (e.g., PVP) or the coating containing the lubricious polymer, to the device or to the intermediate layer(s) already coated on the device. In this capacity, stabilizing polymers may function as a binding agent that reduces the aqueous solubility of the lubricious polymer, while sustaining the coating's lubricity. Stabilizing polymers also may aid in the co-mingling of the different layers, such that there is molecular mingling at the interface between the two layers. Improved binding of the lubricous layer to the device allows for retention of the coating for longer periods of time and ensures that lubricity is maintained. For example, the stabilizing polymer can be a water-insoluble cellulose polymer (e.g., nitrocellulose), polymethylvinylether/maleic anhydride, acrylic or methacrylic polymers, ethylene acrylic acid copolymers, styrene acrylic copolymers, polyvinyl acetals, ethylene vinyl acetate copolymer, polyvinyl acetate, epoxy resins, phenolic resins, copolymers, or nylon, or a combination thereof. These stabilizing polymers may, optionally, be cross-linked. For example, the stabilizing polymer can be a water-insoluble cellulose polymer (e.g., nitrocellulose), polymethylvinylether/maleic anhydride, or nylon, or a combination thereof. In some cases, a water-insoluble cellulose polymer is preferable as a stabilizing polymer, for ease of handling and for tendency to produce coatings with greater long-term wet abrasion resistance than coatings prepared with other stabilizing polymers. When the stabilizing polymer is nitrocellulose, a plasticizing agent can be used in conjunction with the nitrocellulose.

The external layer may include other types of materials to form lubricious layers. Representative examples of compositions that can be used to form a lubricious coating include silicon based polymers, hyaluronic acid, poly(acrylates), polyether polyurethanes, and others known to those skilled in the art. Additional examples may be seen in International Application No. ______, titled “LUBRICIOUS COATINGS” and filed Jan. 31, 2007, which is incorporated by reference in its entirety.

The external layer can additionally include materials such as other polymers, plasticizers, reactive agents such as anti-infective materials, colorants such as dyes and pigments, and the like.

In some circumstances, one or more layers includes an isocyanate. The isocyanate can be, for example, a polyether isocyanate, an isophorone diisocyanate (IPDI), a methylene diphenyl diisocyanate (MDI), a hexamethylene diisocyanate (HDI), or a toluene diisocyanate (TDI).

An exemplary solution for applying an external layer to a surface can include PVP in a range from 0.01% to 30% w/w of the coating solution polymer component, preferably from 0.5 to 20% w/w, and more preferably 1% to 8% w/w. The amount of stabilizer polymer can range from 0.01% to 20% w/w, preferably from 0.05% to 10% w/w, and more preferably 0.01 to 5% w/w. Preferred commercial sources of the pyrrolidone include International Speciality Products (ISP). Preferred commercial sources of the stabilizer include Hagedorn Akteingesellschaft Chemical. Useful ratios of polyvinylpyrrolidone to stabilizing polymer range from 0.04/99.96 to 99.97/0.03 in the coating solutions.

The solution which forms the external layer can be applied to a deposited coating formed from a mixture of polyurethane or polycarbonate-based polyurethane and stabilizer such as cellulose nitrates.

In a solution used for preparing an intermediate layer, the amount of polyurethane or polycarbonate-based polyurethane can range from 0.05% to 40%, preferably from 0.1% to 20%, and most preferably 3% to 12%. The amount of stabilizer polymer can range from 0.1% to 10%, preferably from 0.5% to 7%, and most preferably 1% to 5%. Polyvinylpyrrolodone is available from BASF and ISP in various molecular weight grades. Preferred commercial sources of the polyurethane and polycarbonate-based polyurethane include Cardiotech International and Thermedics, Inc. Preferred commercial sources of the stabilizer include Hagedorn Akteingesellschaft, I.C.I. and Nobel Enterprises. Cellulose nitrates are available in various viscosity and nitration grades from Hagedorn Akteingesellschaft.

In some cases, a primer layer may be applied directly to a relatively inert medical device/instrument surface that lacks reactive functional groups. Either the solution which forms the external layer or an intermediate layer can be applied to the primer. In a solution for applying a primer layer, the amount of primer polymer may range from 0.5% to 6% w/v, preferably from 1% to 4% w/v, and most preferably 1.5% to 3% w/v. The amount of stabilizer polymer can range from 0% to 10% w/v, preferably from 0.2% to 6% w/v, and most preferably 0.3% to 3% w/v. See, for example, U.S. Pat. No. 6,306,176, which is incorporated by reference in its entirety.

Any of the foregoing coating compositions may contain stabilizers, plasticizers, fillers, and the like ranging from, for example, 0.01% to 70% weight %. In addition, these coating compositions may contain crosslinking agents, in amount ranging from 0.01% to 30% weight %.

The lubricious external layer can be formed on an intermediate layer on a surface of the medical device, the intermediate layer being an adherent, flexible hydrogel coating, e.g., as described in U.S. Pat. Nos. 5,997,517, 6,306,176, and 6,110,483, issued to Whitbourne, et al., each of which is herein incorporated by reference in its entirety. The surface of the medical device may be coated with a coating that includes: (a) a stabilizing polymer such as, for example, optionally crosslinked acrylic or methacrylic polymers, ethylene acrylic acid copolymers, styrene acrylic copolymers, polyvinyl acetals, ethylene vinyl acetate copolymer, polyvinyl acetate, epoxy resins, amino resins, phenolic resins, copolymers thereof, and combinations thereof; and (b) an active agent which can be, for example, a hydrophilic polymer selected to interact with the stabilizing polymer so as to produce a lubricious hydrogel.

The surface of the medical device can be an inert surface, e.g., one that is substantially free of reactive functional groups. The inert surface can be modified by a biocompatible surface coating that includes: (a) an intermediate layer having a thickness below about 100 microns such that the intermediate layer does not penetrate more than superficially into the device. The intermediate layer can include at least one bonding polymer bonded non-covalently with the inert surface of the device, and the intermediate layer can include a cross-linked matrix. The biocompatible surface coating can further include (b) an external layer applied to the intermediate layer that adheres to the intermediate layer, the coating remaining adherent to the surface and resistant to abrasion and to removal from the device after soaking in water relative to a coating without the intermediate layer.

The medical device can be coated with an echogenic coating layer. A medical device for inserting into a non-gas target medium can be ultrasonically visible. In particular, the device can include an echogenic structure, e.g., a substrate and (a) a surface having structures (e.g., holes or voids) open to the target medium and able to entrap and retain gas when the device is placed into the target medium, and/or (b) a matrix comprising at or near the surface a non-gas material (for example, a fluid containing microparticle) that changes phase to produce gas bubbles when heated, the entrapped or heated gas causing the device to be ultrasonically visible in the target medium. Echogenic coatings (i.e., coatings including echogenic structures) that may be applied to medical devices include those described in commonly owned U.S. Pat. Nos. 6,610,016 and 6,106,473, issued to Violante et al., and U.S. Patent Application No. 2004/0077948, to Violante et al., each of which is herein incorporated by reference in its entirety.

The compositions and/or solutions used to coat the medical devices can be used on mesh, wiry, porous, non-porous, flat and/or sharp surfaces, whether made of metal, ceramic or polymeric substrates or other surfaces that may be used in medical devices.

The coating can have beneficial characteristics for use on the surfaces of devices such as biomedical implants. The coating can be hydrophilic, absorbing water and swelling in an aqueous environment to become a hydrogel. The coating can have lubricant properties, and can be significantly more slippery when wet than when dry. Instruments coated with the described coatings decrease the amount of frictional force required to penetrate tissue. Thus, medical devices coated with the described lubricious compositions are capable of penetrating into or through tissue more easily than a comparable uncoated instrument. The coating can be thin, e.g., on the order of magnitude of one thousandth of an inch. The coating can be coherent and resistant to removal by wet abrasion, and can adhere to a wide variety of substrates. The coating employs biocompatible substances that are neither toxic nor irritating. The functional characteristics of the coating may be varied as appropriate for many different applications.

Medical devices may be coated with a combination of a coating that imparts echogenicity to the implant and a coating that enhances the lubricity of the device. For example, a medical device (e.g., catheter, biopsy needle, or trocar) may be coated with a coating solution that contains a hydrophilic polymer and then overcoated with a coating that provides an echogenic structure. In another example, a medical device may first be coated with a coating having an echogenic structure (e.g., a coating that contains a plurality of cavities or bubbles and/or echogenic microparticles). Coatings that are both echogenic and lubricous also may be prepared using the compositions described herein. For example, echogenic microparticles may be contained in the lubricious layer of a coated medical device.

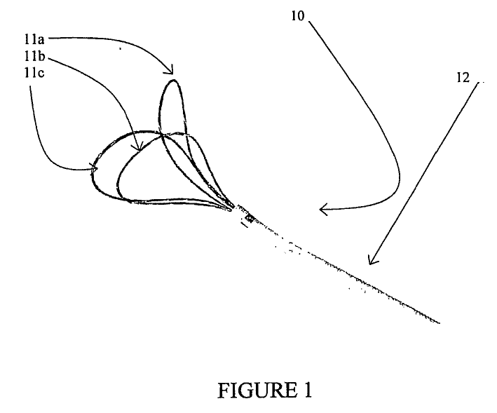

The coating can contain dyes, stains, or pigments or salts useful in diagnosis. The structure of FIGS. 1 and 2 can be coated. See, for example, WO 2005/110302, which is incorporated by reference in its entirety. Referring to FIG. 1, marking knife 10 includes a handle 20 and a cutting portion 30. Referring now to FIG. 2, the cutting portion 30 includes a cutting blade 32 which is held in a retaining means 34, such as a tube, sheath or slot that may be integrally formed with the handle 20 or attached thereto as a separate structure. The blade 32 may be permanently mounted into the retaining means 34 or may be frictionally fit if the remainder of the knife 10 is intended for more than one use. One will also note in FIG. 2 that the retaining means 34 may be formed such that it presents the cutting blade 30 at an angle away from being perpendicular with the handle 20.

Blade 32 may be formed in any one of the conventional shapes known in the art such as a flat blade or a multi-surface blade (as is shown in FIG. 2) and may be curved or rounded. The proximal end of the blade 32 can terminate with a shoulder 36. The shoulder can be relatively flat and perpendicular to the cutting edge of the blade 32. It is contemplated that the shoulder 36 may have a different geometry, such as being convex or concave, depending on the intended use of the surgical knife 10. A bioreactive stain or dye 38 dissolved or dispersed in the coating is placed onto the shoulder 36. The stain can be placed on the shoulder 36 during manufacture and is either provided in a dry form dried directly on the shoulder 36 whereby the stain is otherwise stable until hydrated. Such bioreactive stains or dyes include, but are not limited to, Gentian violet, Indocyanine green, Methylene blue, Cresyl blue, VisionBlue or Trypan blue.

The composition of the coating can be varied to control lubricity, swelling, flexibility, and resistance to removal by wet abrasion. These characteristics of the coating can thus be adjusted for various substrates and applications. The solutions used to prepare the invention can have good shelf stability and remain substantially free of precipitate for periods in the range of months or years, so that various mixtures of the solutions for coatings may be prepared at one time and used to coat substrates later. Alternatively, the hydrophilic and stabilizing polymers, and, if desired, a plasticizing agent and an adherent polymer, may even be prepared in a single solution. Furthermore, because the use of chlorinated solvents or other acute toxics is not required, fewer precautions are necessary to protect workers from health hazards.

The stabilizing polymers, particularly modified cellulose polymers, can be used to make hydrophilic polymers, such as PVP and PVP-vinyl acetate copolymers, stable and insoluble in water and lower alcohols. The resulting combination, when applied to a substrate, produces a coating that has a stable layer or layers that are bonded to a substrate surface, that is not slippery when dry but is desirably lubricious when wet, and is resistant to removal by conditions of wet abrasion. The coating layer bonds to an impervious surface such as stainless steel or glass. It also bonds to polymer surfaces where the surface interacts with the components of the coating.

Preferably, the solution for depositing the external layer includes a solvent that is capable of solubilizing (at least partially) components of the external layer and an intermediate layer. As such, the solvent can promote penetration of the external layer components into the intermediate layer, and is believed to bring about a mixing of the components of both layers.

Such mixing can facilitate chemical reactions such as cross-linking between the components, or facilitate physical mixing of layers without chemical reactions. In some embodiments, there can be a high degree of cross-linking or intermolecular mingling between a hydrophilic polymer and a stabilizing polymer at the interface between the external and intermediate layers of the coating. Thus, a region between the two layers may be created as a result of cross-linking or intermolecular mingling between the polymers contained in the separate layers. The slight degree of cross-linking or mingling at the outer surface of the coating can aid in providing the lubricity of the coating.

In practice, the composition of the solvent mixture can be adjusted so that the degree of penetration of the external layer into the intermediate layer is in a useful range. For example, if the external layer solvent mixture is too active toward the intermediate layer, then too much penetration into the intermediate layer occurs, and the external layer may be rendered less lubricious when wet than desired. Conversely, if the external layer solvent is too inactive toward the intermediate layer, then too little penetration of the external layer into the intermediate layer occurs, and the coating can be too easily removed from the inner layer by wet abrasion.

An exemplary coating can be applied to the surface of a medical device with sufficient thickness and permanence to retain the coating's desirable qualities throughout the useful life of the coated device. The coatings are desirably non-reactive with living tissue and may be non-thrombogenic in blood.

When tested by subjective methods, the coatings when wet are more slippery than wet, greased glass, and, when dry, are no more slippery than dry glass. The coatings are resistant to removal by wet abrasion as determined by running water over the coatings and rubbing between tightly gripped fingers while wet. The coatings have high adherence when dry, as determined by attaching adhesive tape, pulling the tape off with a vigorous action, and then wetting the coated substrate to determine whether the taped portion retained the lubricant coating. The coatings remain adherent and coherent for extended periods when stored in water, and neither peel off, dissolve, nor dissociate.

Other approaches to produce adherent coatings on difficult-to-coat substrates such as metals (e.g., stainless steel or titanium) can include using a primer layer.

Adherent coatings may be prepared using plasma treatment prior to coating. Plasma treatment (e.g., oxygen or nitrogen plasma) may also be used to introduce functional groups on the surface which may further improve adhesion of the coating to the device.

In an exemplary embodiment, a coated medical device, e.g., a knife or scalpel, is removed from its sterile wrapper, is gripped by a user (e.g., surgeon), the blade is dipped in alcohol or water to make the blade slippery, and the blade is used to make an incision while the blade is wet. In another exemplary embodiment, a coated medical device, such as an intravascular retrieval snare system, is removed from its sterile wrapper, is gripped by a user (e.g., surgeon), the snare is dipped in alcohol or water to make the coating slippery, and the snare is placed into a vein or artery and monitored by ultrasound imaging. The snare is then used to retrieve foreign objects from the patient's vascular system. In other embodiments, the blade or snare is provided in a wetted state, and does not need to be wetted by a user before use.

In some embodiments, tools or devices useful in surgical procedures, such as dissection, curetting, suturing, and cutting with scissors, a surgical knife, blade, or a scalpel, are provided with a lubricious and optionally echogenic coating. The surgical instruments may include rongeurs, dissectors, knives, scalpels, scissors, needle drivers, suture holders, curettes, electrodes, probes, forceps, aneurysm clip applicators, and the like. The surgical instruments can be scalpels and knives (e.g., opthalmology knives). For example, the surgical knife may be a SHARPOINT ophthalmic blade.

The surgical instruments can be catheters, guide wires, or medical tools. Catheters include, for example, PTCA catheters, cardiology catheters, central venous catheters, urinary catheters, drain catheters, and dialysis catheters. The catheter may be a percutaneous biliary drainage catheter which may come in different lengths for the biliary procedure, one of which reaches the duodenum for correct placement of a guide wire, and may further include a locking pigtail. Other exemplary catheters include a nephrostomy catheter, of e.g. soft polyurethane for optimal kink resistance, with or without plastic and metal stiffener, with or without large drainage holes to provide minimal tissue trauma and ulcerations, and thereby reduce patient discomfort. The surgical instruments can be connecting tubes for drainage bags. Guidewires, as well as tips of wire guides used in conjunction with, e.g., catheters, can be coated.

The surgical instruments can be introducer sets, which for catheters include a co-axial system for placement of a guide wire in non-vascular procedures. The system includes a coaxial dilator (which can be coated or uncoated) and at least one guide wire, which can be coated or uncoated. Guide wires include CANALIZER wires, from PBN Medicals.

Other catheter/tube medical devices which can be prepared with the coatings include: abdominal cavity drainage, ablation catheters, angiography catheters, angioplasty balloons, arterial line, artificial insemination catheters, Bivona tracheostomy tubes, catheters, cavity drainage catheters, central venous catheters, cholangiography, cutting loops, diagnostic electrode catheters, dilation balloons, drainage catheters, electrode catheters, embolectomy catheters, endobronchial tubes, endotracheal tubes, epidural catheters, Foley catheters, guiding catheters, hemodialysis catheters, Kumpe access catheters, laryngectomy tubes, laser ureteral catheters, pacing catheter, percutaneous access/catheter sets, percutaneous enteral feeding devices, peripheral catheters, PICC lines catheters, pigtail ureteral catheters, rectal pressure catheters, renal access catheter, stimulating catheter, suction catheters, thermodilution intra-aortic balloon catheters, tracheostomy tubes, ureteral drainage, urinary catheters, urodynamics catheters, or wedge pressure catheters.

Surgical needles and sutures can be coated. Sutures and suture needles (which may be attached to the suture) may be coated with a lubricious and/or echogenic coating as described herein. The entire needle or suture or only a portion of the needle or suture may be coated. It may be practical to coat just the distal ⅔ of a suture needle leaving the proximal end uncoated, such that it is more easy to grip by the surgeon during use. Among the needles that can be coated are needles of between ⅛ and ½ circle with dimensions of between 1 and 100 mm, for example, between 5 and 50 mm. The needle can be, for example, a side cutting lancet needle; a reverse cutting needle; a precision reverse cutting needle; an ULTRAGLIDE needle; or a DERMAGLIDE needle. More particularly, coated needles can be of ⅛ circle having dimensions of 5.51 or 14.99 mm.; needles of ⅜ circle having dimensions of 6.15, 6.6, 6.15, 6.68, 11, 12, 13, 14, 16, 18, 24, 30, 36 or 40 mm; ¼ circle needles with dimensions of 6.6, 8, or 8.51 mm; needles of ½ circles with dimensions of 9, 16, 15, 16, 18, 20, 24, 26, 27, 37, or 40 mm; or bicurve needles of 4.8 or 5.51 mm. Other needles used in medical application which can be produced include: amniocentesis needles, brachytherapy needles, core tissue biopsy needles, docking needle discograms, epidural needles, fascial incision needles, Huber needles, insulin pump needles, lumbar puncture needles, nerve block needles, procedural needles, prostate biopsy needles, or vertebroplasty needles.

Cutting instruments, such as scalpels, knives and scissors, whether disposable or not, and other cutting devices may be coated. Suitable coated cutting instruments include micro-blade; slit knives (in which the blade is optionally angled) with a knife size of 2.5 to 5.0 mm; stab knives; incisional instruments; sideport knives; surgical blades including carbon steel blades; stainless steel blades; surgical knives and microsurgical knives; scalpels; safety scalpels blade remover; scalpel cartridges; shave biopsy devices; or safety prep razors. In an exemplary embodiment, the surgical instrument may be a precision knife for micro-incisions. The blade of the instrument may comprise about 1.3 mm to about 1.6 mm in width. The dimensions of the blade may be 1.5 mm×1.7 mm, 1.5 mm×2.0 mm or 1.7 mm×2.0 mm. In some embodiments, the blade may be in sizes of about 0.6 mm, 0.8 mm, or 1.1 mm. Other cutting instruments that can be coated include: dissection knives, electrosurgical bipolar, corneal blades, or corneal punches. The blade of a surgical instrument may be flat, have different geometrical shapes and/or featuring tapered facets.

In some embodiments, the entire surface of the blade may be coated with one or more of the described coatings. Alternatively, the coating is applied to only the tip or blade of the surgical instrument. There may be a number of different configurations of working tips or blades such as round knife blades, probes, blunt blades, curved blades, suture holders, needle holders, scissors, curettes and the like. The coating may be applied to only the cutting edge of the instrument.

In another exemplary embodiment the coating is applied to the tip of the surgical instrument only or to the whole snare including the catheter portion. Other instruments providing a substrate treatment in accordance with the invention include condoms, contact lenses, peristaltic pump chambers, arteriovenous shunts, gastroenteric feed tubes and endotracheal tubes, or other implants of metal or polymer substrate.

Other medical equipment which can be produced include: angiography, balloon stents, bone biopsy device, cement delivery system, electrosurgical electrodes, embryo replacement, guidewires, hemodialysis products, joint anchors interference, screws & fixation, neuro micro-driver, osteo intoducer, percutaneous sheath introducers, peripheral nerve block (PNB), punctum plugs, RF thermoablation, Roadrunner PC wire guides, spinal fixation, spinal implants, spinal screws, stone baskets, thoracentesis, ureteral stents, urethral dilation, uretral stents, wire guides, bone cement delivery device, bone filler device, bone tamp, cystome, pacing electrodes, paracentesis, stone manipulating devices, two part trocar sets, braided sutures, or endoscopic suturing devices.

Sutures

The coatings can be applied to a surface of a suture, for example a silk microsuture, e.g., one having a diameter of less than 0.5 mm, 0.4 mm or less, 0.3 mm or less, or 0.2 mm or less. The sutures can be monofilamentary or multifilamentary, and can be plain or self-retaining, the self-retaining sutures having retainers (such as barbs or other tissue gripping structures) that engage when the suture is pulled in a direction other than that in which it was deployed in the tissue. A self-retaining suture may be unidirectional, having retainers oriented in one direction along the suture body, or bidirectional, having one of more retainers near one of the suture oriented in one direction and one or more retainers near the other end of the suture oriented in the opposite direction. Retainers may be configured to have tissue insertion points (such as barbs), tissue insertion edges (such as conical or frusto-conical structures), etc., and can help to anchor the sutures in place once introduced by a surgeon. The suture can be introduced so that the retainers do not engage while the suture is being placed. With a self-retaining suture, the coating can be only on an exterior surface of a retainer. For example, the coating can be applied to the suture before the retainers are formed, so that when the retainers engage, the engaging surface is substantially free of the coating. In this way, tissue being sutured contacts a lubricious surface of the suture as the suture is introduced, but when the retainer engages, a non-coated surface of the retainer contacts the tissue. The non-coated surface of the retainer can provide greater friction to the tissue than the coated surfaces of the suture, providing additional anchoring.

The lubricious and/or echogenic coatings may be applied to various types of sutures. The sutures may be absorbable (e.g., those that are degraded by the body's enzymatic pathways and generally lose tensile strength by 60 days after implantation), and may be made of polymers or copolymers of glycolic and lactic acid. Exemplary absorbable sutures include catgut (both plain and chromic) (e.g., those with a trade name PROGUT from Dolphin Sutures, India), and those derived from polyglycolic acid with a trade name PETCRYL (Dolphin Sutures, India) and with a trade name DEXON™ (Sherwood Services AG, Schaffhausen, Switzerland), from poliglecaprone 25 with a trade name MONOCRYL (copolymer of about 75% glycolide and about 25% caprolactone, Johnson & Johnson Co., New Brunswick, N.J.), from polyglactin 910 (such as VICRYL, coated VICRYL, coated VICRYL Plus Antibacterial sutures that contain antibacterial triclosan, and Coated VICRYL RAPIDE sutures, Johnson & Johnson Co., New Brunswick, N.J.), MULTIPASS Needle Coating (Johnson & Johnson Co., New Brunswick, N.J.), copolymer of about 67% glycolide and about 33% trimethylene carbonate sold as MAXON™, Wyeth, Madison, N.J., and from polydioxanone with a trade name PDS II (Johnson & Johnson Co., New Brunswick, N.J.).

In addition to the sutures described above, degradable sutures can be made from polymers such as polyglycolic acid, copolymers of glycolide and lactide, copolymers of trimethylene carbonate and glycolide with diethylene glycol (e.g., MAXON™, Tyco Healthcare Group), terpolymer composed of glycolide, trimethylene carbonate, and dioxanone (e.g., BIOSYN™ [glycolide (60%), trimethylene carbonate (26%), and dioxanone (14%)], Tyco Healthcare Group), copolymers of glycolide, caprolactone, trimethylene carbonate, and lactide (e.g., CAPROSYN™, Tyco Healthcare Group). These sutures can be in either a braided multifilament form or a monofilament form. The polymers can be linear polymers, branched polymers or multi-axial polymers. Examples of multi-axial polymers used in sutures are described in U.S. Patent Application Publication Nos. 20020161168, 20040024169, and 20040116620, each of which is incorporated by reference in its entirety.

Absorbable sutures may be used below the surface of the skin to provide support to the skin closure. They may also be used in areas where suture removal might jeopardize the repair such as with small children who might not easily cooperate with suture removal.

Sutures on which the lubricious and/or echogenic coatings may be applied may also be non-absorbable. Non-absorbable sutures are permanent and include sutures made of polyamide (also known as nylon, such as nylon 6 and nylon 6.6), polyester (e.g., polyethylene terephthlate), polytetrafluoroethylene (e.g., expanded polytetrafluoroethylene), polyether-ester such as polybutester (block copolymer of butylene terephthalate and polytetra methylene ether glycol), polyurethane, metal alloys, metal (e.g., stainless steel wire), polypropylene, polyethelene, silk, and cotton. Exemplary non-absorbable sutures include coated polyester sutres with a trade name Procare (Dolphin Sutures, India), GORTEX™ (made of expanded polytetrafluoroethylene, sold by Gore), NOVAFIL™ (made of polybutester, Wyeth, Madison, N.J.), monofilament polyamide sutures with a trade name Linex (Dolphin Sutures, India), SUTURA (black braided silk sutures, Sutura Inc., Fountain Valley, Calif.), monofilament polypropylene sutures with a trade name Duracare (Dolphin Sutures, India), MONOSOF (monofilament nylon suture, United States Surgical Co., Norwalk, Conn.), DERMALON™ (monofilament nylon suture, Sherwood Services AG, Switzerland), SURGILON™ (braided nylon suture coated with silicone, Sherwood Services AG, Switzerland), Ethilon nylon suture (Ethicon, Inc., Somerville, N.J.), ETHIBOND EXCEL (braided polyester suture from Johnson & Johnson Co., New Brunswick, N.J.), Pronova poly(hexafluoropropylene-VDF) suture (Ethicon, Inc. Somerville, N.J.), TEVDEK™ (braided polyester suture from J.A. Deknatel and Son, Inc. New York, N.Y.), PROLENE™ (polypropylene suture from Ethicon, Inc., Somerville, N.J.), FLUOROFIL™ (polypropylene suture from Pitman-Moore, Inc. Lake Forest, Ill.), and MERSILENE™ (polyester fiber suture from Ethicon, Inc., Somerville, N.J.).

Additional exemplary sutures to which the lubricious and/or echogenic coatings may be applied are various sutures available from Surgical Specialities Co., Reading, Pa.), including monoderm undyed or dyed monofilament sutures, clear or dyed PCL monofilament sutures, dyed polypropylene monofilament sutures, undyed braided POLYSYN FA sutures, dyed or undyed braided PGA sutures, dyed or undyed braided polysyn suture, dyed monofilament polysyn sutures, dyed braided polyester sutures, braided silk sutures, dyed braided polyviolene sutures, plain or chromic gut sutures, dyed or undyed monofilament nylon sutures, and dyed pliable nylon sutures.

Additional exemplary sutures to which the lubricious and/or echogenic coatings may be applied are various sutures available from Tyco International Ltd., Bermuda or its companies. Such sutures include SURGITIE™ (single use ligating loops with delivery system) and SURGIWIP™ (single use sutrue ligatures with delivery system), absorbable sutures such as POLYSORB™ (suturtes composed of LACTOMER™ glycolide/lactide copolymer, a synthetic polyester composed of glycolide and lactide (derived from glycolic and lactic acids), DEXON™ II (synthetic suture composed of homopolymer of glycolic acid and coated with POLYCAPROLATE™, a copolymer of glycolide and epsilon-caprolactone), DEXON™ S (synthetic sutures composed of the homopolymer of glycolic acid), MAXON™ CV (polyglyconate synthetic sutures prepared from a copolymer of glycolic acid and trimethylene carbonate), plain, mild chromic, and chromic gut sutures composed of purified connective tissue (mostly collagen) derived from the serosal layer of beef intestines, and non-absorbable sutures such as DERMALON (nylon), MONOSOF (nylon), SURGILON (nylon), SURGIDAC™ (polyethylene terephthalate), TI-CRON™ (sutures prepared from fibers of high molecular weight, long chain and linear polyesters having recurrent aromatic rings as an intergral component), SURGIPRO™ (sutures composed of an isotactic crystalline steroisomer of polypropylene (a synthetic linear polyolefin) and polyethylene), SURGIPRO™ II (sutures composed of an isotactic crystalline steroisomer of polypropylene (a synthetic linear polyolefin) and polyethylene), NOVAFIL™ (sutures composed of polybutester, a copolymer of butylenes terephthalate and polytetramethylene ether glycol), VASCUFIL™ (sutures composed of a copolymer of butylenes terephthalate and polytetramethylene ether glycol and coated with POLYTRIBOLATE™, an absorbable polymer of ε-caprolactone/glycolide/poloxamer 188), FLEXON™ (twisted multistrand steel sutures coated with orange or white PTFE poly(tetrafloroproethylene) or clear FEP poly(tetrafluoroethylene-co-hexafluoropropylene), SOFSILK™ (sutures composed of natural proteinaceous silk fibers that are treated to remove the naturally-occurring sericin gum), and stainless steel sutures.

In certain embodiments, sutures to which the lubricious and/or echogenic coatings may be applied are used for joining tissue in surgical procedures including, without limitation, joining and holding closed a wound (such as a surgical incision) in bodily tissue, fastening junctions of wounds, tying off wounds, and joining a foreign element to tissue.

In certain embodiments, sutures to which the lubricious and/or echogenic coatings may be applied are used in various dental procedures, i.e., oral and maxillofacial surgical procedures, and thus may be referred to as “dental sutures.” The above-mentioned procedures include, but are not limited to, oral surgery (e.g., removal of impacted or broken teeth), surgery to provide bone augmentation, surgery to repair dentofacial deformities, repair following trauma (e.g., facial bone fractures and injuries), surgical treatment of odontogenic and non-odontogenic tumors, reconstructive surgeries, repair of cleft lip or cleft palate, congenital craniofacial deformities, and esthetic facial surgery. Many of the various sutures described above are used in such procedures and are available from many of the same commercial sources. As above, dental sutures may be degradable or non-degradable. Sutures used in oral and maxillofacial surgical procedures may typically range in size from USP 2-0 to USP 6-0. Dental sutures may have a surgical needle attached.

In certain embodiments, self-retaining sutures to which the lubricious and/or echogenic coatings may be applied are used in tissue repositioning surgical procedures. Such surgical procedures include, without limitation, face lifts, neck lifts, brow lifts, thigh lifts, and breast lifts. Self-retaining sutures used in tissue repositioning procedures may vary depending on the tissue being repositioned; for example, sutures with larger and further spaced-apart retainers may be suitably employed with relatively soft tissues such as fatty tissues.

In certain embodiments, sutures to which the lubricious and/or echogenic coatings may be applied are microsutures. Microsutures are used in microsurgical procedures that are performed under a surgical microscope. Such surgical procedures include, but are not limited to, reattachment and repair of peripheral nerves, spinal microsurgery, microsurgery of the hand, various plastic microsurgical procedures (e.g., facial reconstruction), microsurgery of the male or female reproductive systems, and various types of reconstructive microsurgery. Microsurgical reconstruction is used for complex reconstructive surgery problems-when other options such as primary closure, healing by secondary intention, skin grafting, local flap transfer, and distant flap transfer are not adequate. Microsutures are available from many of the commercial sources identified above and are made from the same materials described above. As above, microsutures may be degradable or non-degradable. Microsutures have a very small caliber, often as small as USP 9-0 or USP 10-0, and may have an attached needle of corresponding size.

Additional exemplary sutures to which the lubricious and/or echogenic coatings may be applied are described in U.S. Pat. Nos. 5,766,188, 4,441,496, 6,692,516, 4,550,730, 4,052,988, and U.S. Patent Application Publication Nos. 2005267532, 2005240224, 2004111116, 2004088003, 2002095180, each of which is incorporated by reference in its entirety.

Sutures to which the lubricious and/or echogenic coatings may be applied may be commercially available or may be made using any suitable method, including injection molding, stamping, cutting, laser, extrusion, separate manufacture and subsequent attachment of retainers, and the like. With respect to cutting, polymeric thread or filaments may be purchased, and retainers subsequently cut or added onto the filament body. In certain embodiments, barbed sutures may be produced according to U.S. Pat. No. 6,848,152 and U.S. Patent Application Publication Nos. US 2004/0226427 and US 2004/0060409, each of which is incorporated by reference in its entirety.

In certain embodiments, sutures to which the lubricious and/or echogenic coatings may be applied are already attached to surgical needles. Attachment of sutures and surgical needles is described in U.S. Pat. Nos. 3,981,307, 5,084,063, 5,102,418, 5,123,911, 5,500,991, 5,722,991, 6,012,216, and 6,163,948, and U.S. Patent Application Publication No. US 2004/0088003, each of which is incorporated by reference in its entirety. A method for the manufacture of surgical needles is described in U.S. Pat. No. 5,533,982, and a method for the manufacture of polymer-coated surgical needles is described in U.S. Pat. No. 5,258,013, each of which is incorporated by reference in its entirety.

In certain embodiments, the sutures to which the lubricious and/or echogenic coatings may be applied are pointing at both ends (including suture connectors as described in U.S. Pat. No. 6,241,747, which is incorporated by reference in its entirety). In certain other embodiments, the sutures may have one pointing end and an anchor on the other end. The anchor may be used to secure the implantation of the suture in soft tissue (e.g., those described in U.S. Patent Application Publication No. US2005/0267531, which is incorporated by reference in its entirety) or the attachment of sutures to the bone (e.g., those described in U.S. Pat. No. 6,773,450 and PCT Application Publication No. WO 2004/014236, each of which is incorporated by reference in its entirety).

In certain other embodiments, the suture may be a relatively short suture with sharp pointing ends. Such a suture may function similar to a staple when used in connecting tissues and thus permits a surgeon to rapidly and securely attach the edges of a wound in a bodily tissue or reconfigure the tissue without the necessity for threading and tying numerous individual stitches or for the use of a complicated tool to insert the suture. This type of sutures may thus be referred to as “suture connector.” In certain embodiments, the suture connector may be a bi-directional self-retaining suture. In certain other embodiments, the suture connector may be found by linking two relatively short uni-directional self-retaining sutures together to form a bi-directional self-retaining suture (see, U.S. Pat. No. 6,241,747, which is incorporated by reference in its entirety).

Catheters

The lubricious and/or echogenic coating can be applied to a surface of a catheter or a catheter accessory, such as, for example, a catheter patency device, a centesis catheter, a drainage catheter, one or more components of a guidewire introduction system, one or more components of a hystero-access catheter set, or a vessel sizing catheter.

A centesis catheter can have a variety of dimensions, e.g., from 4 F to 5 F×7 cm to 20 cm. In particular, a centesis catheter can have dimensions of 4 F×7 cm, 4 F×10 cm, 4 F×15 cm, 5 F×7 cm, 5 F×10 cm, 5 F×15 cm, or 5 F×20 cm. The centesis catheter can have four distal side holes to provide drainage in small cavities, and can have a Luer lock hub for secure, one-handed placement. Exemplary centesis catheters include SKATERS® centesis catheters and from InterV.

The drainage catheter can have a variety of dimensions, e.g., from 6 F to 16 F×20 cm to 25 cm. In particular, a drainage catheter can be a 6 F×20 cm locking pigtail catheter that accepts a 0.035″ guide wire, a 7 F×20 cm locking pigtail catheter that accepts a 0.035″ guide wire, a 8 F×25 cm locking pigtail catheter that accepts a 0.038″ guide wire, a 10 F×25 cm locking pigtail catheter that accepts a 0.038″ guide wire, a 12 F×25 cm locking pigtail catheter that accepts a 0.038″ guide wire, a 14 F×25 cm locking pigtail catheter that accepts a 0.038″ guide wire, a 16 F×25 cm locking pigtail catheter that accepts a 0.038″ guide wire, a 6 F×20 cm non-locking pigtail catheter that accepts a 0.035″ guide wire, or a 7 F×20 cm non-locking pigtail catheter that accepts a 0.035″ guide wire. The catheter can be used with Seldinger or Trocar insertion techniques. Exemplary drainage catheters include SKATER® single step catheters and SKATERS® drainage catheters from InterV.

The drainage tubing portion of the catheter can be made of a thermoplastic polyurethane elastomer such as pellethane. This portion can be spray-coated with a solution and dried to coat the drainage tubing portion of the catheter with a lubricious coating. In one embodiment, the solution used for spray coating has the composition shown in the following table.

| Component | Weight % |
|---|---|
| Denatured Anhydrous Ethanol (EtOH) | 10.10 |
| Benzyl Alcohol | 18.10 |
| Cyclohexanone | 47.16 |
| Tetrahydrofuran (THF) | 22.40 |
| Nitrocellulose stock solution | 0.14 |
| Polyvinylpyrrolidone K-90 (PVP K-90) | 2.10 |

Nitrocellulose Stock Solution

| Component | Weight % |
|---|---|
| ¼ RS (H27) Nitrocellulose (Manufacturer: Hagedorn) | 9.00 |
| 4-butyrolactone (BLO) | 91.00 |

Coated drainage catheters can be sterilized by treatment with ethylene oxide.

A hystero-access catheter set can include several components for use together during a medical procedure, e.g., selective salpingography or fallopian tube procedures. For example, the set can include a 10 F hystero-access balloon catheter (which can have a non-latex balloon for sealing the cervix), and a 5 F or 7 F selective salpingography catheter, which can accept a 0.035″ guidewire or 3 F catheter for coaxial introduction. The set can include a 0.035″ guidewire. One or more of the components of the hystero-access catheter set can have a surface coated with a lubricious coating.

A vessel sizing catheter can be, for example, a 5 F×90 cm pigtail catheter that accepts a 0.035″ guide wire and has 6 side holes; or a 5 F×65 cm straight catheter that accepts a 0.035″ guide wire and has 10 side holes. Either can include radiopaque bands (e.g., gold bands) at regular intervals for measurement, e.g., during angioplasty. Exemplary vessel sizing catheters include GOLDEN-RULES vessel sizing catheters from InterV.

Needles

A variety of needles and accessories can advantageously include a surface having a lubricious and/or echogenic coating. For example, the needle can be a biopsy site marker, a bone marrow biopsy needle, a core biopsy replacement needle, a breast localization needle, a component of a galactography kit, an injection needle, a soft tissue biopsy coaxial introducer needle, or a soft tissue biopsy disposable needle (optionally for use with reusable automatic instruments or semi-automatic instruments).

A biopsy site marker can include both a bio-absorbable plug and a permanent anchor, to permanently mark a breast biopsy site, allowing for future identification of the biopsied area. The highly visible plug portion can be absorbed into the breast tissue, while the anchor remains in place allowing for long-term stability and potentially permanent visualization. The bio-absorbable plug provides ultrasound, MRI, and mammography visibility for up to six weeks, and allows for rapid and accurate marker placement under ultrasound guidance. An exemplary biopsy site marker is the V-MARK® sold by InterV. Echogenic materials and compositions may be coated onto the plug material or may be incorporated directly into the plug material to further enhance visualization under ultrasound.

A bone marrow biopsy needle can have a variety of dimensions, such as, for example, 15 G×2.688″, 15 G×4″, 16 G×2.688″, 16 G×4″, 8 G×4″, 8 G×6″, 11 G×4″, 11 G×6″, 13 G×2″, 13 G×3″, in either I-needle or J-needle form.

A breast localization needle can have a variety of dimensions, such as 20 G×3 cm, 20 G×5 cm, 20 G×7.5 cm, 20 G×10 cm, or 20 G×12.5 cm. The needle can have optionally have a J-shape, and optionally include centimeter markings for depth measurement. The needle can include a side barb and may be flexible or rigid.

A galactography kit can include an injection cannula (e.g., a 24 G or 30 G curved injection cannula) with dilator (e.g., a 0.010 or 0.012 diameter dilator).

An injection needle can be, for example, a multi-pronged injection needle, which can have multiples tines (each with one or more through-holes) for fluid delivery. The needle can have a trocar-style tip. The needle can be an 18 G needle with a length of, for example, 10 cm, 15 cm or 20 cm. The injection needle can be a QUADRA-FUSE needle sold by InterV.

A soft tissue biopsy coaxial introducer needle can be used with a biopsy instrument and optionally with a biopsy site marker. For example, the introducer needle (e.g., for use with a BioPince® biopsy instrument) can have dimensions of 15 G×6.8 cm, 15 GA×11.8 cm, 17 GA×6.8 cm, 17 GA×11.8 cm, or 17 GA×16.8 cm. The needle can have centimeter markings, and an echogenic tip. Introducer needles for use with SUPERCORE instruments can have dimensions of 13 G×3.9 cm, 13 G×9.9 cm, 15 G×3.9 cm, 15 G×9.9 cm, 17 G×3.9 cm, 17 G×9.9 cm, 17 G×14.9 cm, 19 G×4.2 cm, or 19 G×10.2 cm. Introducer needles for use with TRUCORE instruments can have dimensions of 13 G×5.1 cm, 13 G×11.1 cm, 15 G×5.1 cm, 15 G×11.1 cm, 17 G×5.1 cm, 17 G×11.1 cm, 17 G×15.1 cm, 19 G×5-4 cm, or 19 G×11.4 cm. Introducer needles for use with TRUCORE instruments can have dimensions of 13 G×4.6 cm, 13 G×10.6 cm, 15 G×4.6 cm, 15 G×10.6 cm, 17 G×4.6 cm, 17 G×10.6 cm, 17 G×14.6 cm, 19 G×4.9 cm, or 19 G×10.9 cm. Introducer needles can also be used with PRO-MAG, OSTY-CORE, and ACN biopsy needles.

A soft tissue biopsy disposable needle can be, for example, a MAXICELL needle which can harvest tissue on both forward and backward thrusts of the needle. The needle can have a 30° matched ground needle tip geometry that helps harvest a cluster of intact cells. The needle can have numbered centimeter marks for depth placement. The needle can have an echogenic tip. The needle can have dimensions of 22 G×5 cm, 22 G×9 cm, or 22 G×15 cm. The soft tissue biopsy disposable needle can be used with an introducer needle.

A soft tissue biopsy disposable needle can be a Chiba style, a spinal style, a Franseen style, Westcott style, or a Greene style needle. The needle can have numbered centimeter marks for depth placement. The needle can have an echogenic tip. The needle can have dimensions of, for example, 18 G, 20 G or 22 G, and a length of 9 cm to 20 cm.

A soft tissue biopsy disposable needle can be a TECHNA-CUT needle, which can have a trocar style needle tip that allows for easy direct puncture while minimizing tissue damage, and a precise cutting edge on the outer cannula that contributes to a complete, intact core specimen. A TECHNA-CUT needle can have dimensions of, for example, 16 G to 23 G and a length of 6 cm to 15 cm.

PRO-MAG needles (e.g., for use with PRO-MAG biopsy instruments) can include a 19 mm sample notch, which ensures sufficient tissue for clinical diagnosis. The needle can have numbered centimeter marks for depth placement. The needle can have an echogenic tip. The needle can be used with a coaxial introducer needle. A PRO-MAG needle can have dimensions of, for example, 14 G to 20 G, and a length of 10 cm to 30 cm.

A SUPERCORE needle can include an adjustable specimen notch (exposing either 19 mm or 9.5 mm), to provide clinical flexibility. The needle can have numbered centimeter marks for depth placement. The needle can have an echogenic tip. The needle can be used with a coaxial introducer needle. A SUPERCORE needle can have dimensions of, for example, 14 G to 20 G, and a length of 9 cm to 20 cm.

A disposable soft tissue biopsy needle for use with an automatic instrument can be, for example, a needle for use with a BIO-PINCE instrument, which can include a tri-axial cut and trap cannula system, to cut the specimen and hold it in the cannula. Needles for use with the BIO-PINCE instrument can be 16 G or 18 G with a length of 10 cm to 20 cm.

Vascular Interventional Devices

Tools or devices used during medical procedures for foreign body retrieval and manipulation procedures can include a surface with a lubricious echogenic coating. Such tools or devices include those as used in the cardiovascular system or hollow viscus to retrieve or manipulate foreign objects. The medical devices can be intravascular retrieval snares or baskets and other laparoscopic devices.

For example, the intravascular retrieval snare can be the EN SNARE system. With reference to FIG. 3, the intravascular retrieval snare can include an intravascular retrieval snare system (10), having a tip comprising 3 interlaced loops (11a, 11b and 11c) that enables the capture, retrieval or manipulation of objects in a vascular body. The instrument can be rotated during use to provide positive engagement with targeted objects. The tip of the device may comprise of a variety of materials including metals, such as stainless steel or interwoven platinum strands. In another aspect, the tip loops may comprise super-elastic nitinol wire. The snare system may comprise a guiding catheter (12) and/or an introducer/back loading device and/or a steering handle. In particular, an outer surface of guiding catheter 12 can be coated with a lubricious coating. Alternatively, the coating can be applied to the tip of the surgical instrument only, or to the whole snare including the catheter portion.

The instrument can be a mini snare with a diameter of from about 1 mm to about 50 mm and a length of from about 100 cm to about 200 cm; and about 3 F to about 7 F by about 120 cm to about 150 cm catheter. For example, the instrument may be a mini snare with 2-4 mm diameter×175 cm length and 3 F×150 cm catheter, a mini snare with 4-8 mm diameter×175 cm length and 3 F×150 cm catheter, a standard snare with 6-10 mm diameter×120 cm length and 6 F×100 cm catheter, a standard snare with 9-15 mm diameter×120 cm length and 6 f×100 cm catheter, a standard snare with 12-20 mm diameter×120 cm length and 6 F×100 cm catheter, a standard snare with 18-30 mm diameter×120 cm length and 7 F×100 cm catheter, or a standard snare with 27-45 mm diameter×120 cm length and 7 F×100 cm catheter.

Other intravascular snare devices, which have a variety of sizes and configurations, may be used in conjunction with the coating. Examples of snares are described in U.S. Pat. No. 6,913,612 to Palmer, et al., U.S. Pat. No. 3,828,790 to Curtiss et al., U.S. Pat. No. 5,171,233 to Amplatz et al., U.S. Pat. No. 5,098,440 to Hillstead and U.S. Pat. No. 6,099,534 to Bates, et al. each of which are herein incorporated by reference in their entirety.

Other vascular interventional devices include a non-invasive hemostasis pad or a vascular access set. The hemostasis pad can be, for example, a V-PAD®, for use in the local management of bleeding wounds such as vascular access sites, percutaneous catheters or tubes, surgical debridement and lacerations. The vascular access set can be, for example, a V-STICK vascular access set for placement of 0.035″ or 0.038″ guidewires into the vascular system using small needle access to reduce puncture site and vessel trauma. The set can include a coaxial dilator, (e.g., a 4 F×10 cm or 5 F×10 cm dilator), a 21 G×7 cm needle (optionally with echogenic tip), and a guidewire.

Trocars

The coating can be applied to trocars, such as, for example, a CVP feeding trocar, or a CVA feeding trocar, such as those available from American Medical Instruments, Inc.

Huber Needles in Cannula Form

The coating can be applied to a Huber needle, e.g., in cannula form, straight or bent (e.g., with a 90° bend) for use in continuous, portal, and intravenous drug therapy. Suitable Huber needles are available from American Medical Instruments, Inc.

Epidural Needles

The coating can be applied to an epidural needle, such as a Tuohy or Hustead epidural needles as well as side port pencil point and standard spinal needles. Suitable epidural needles are available from American Medical Instruments, Inc.

Repositionable Localization Needles

The coating can be applied to a repositionable localization needle (e.g., for use in conjunction with mammography). The repositionable localization needle can be, e.g., a Hawkins I or Hawkins II needle, or a Homer Mammalok® needle from InterV.

A Hawkins I needle can include a retractable side barb to lock the needle in place, or, when retracted, to allow repositioning. The needle can include centimeter markings for depth placement, and a lock-down disk to stabilize the needle. The needle can be a 20 gauge needle with a length ranging from 5 cm to 12.5 cm.

A Hawkins II needle can be a traditional “hardwire” needle, or a “cable,” i.e., a strand of smaller wires, which can provide greater flexibility. The needle can include markings for depth placement, a skin retention clip to stabilize the needle. The needle can be a 20 gauge needle with a length ranging from 5 cm to 12.5 cm.

A Homer Mammalok® needle can have a retractable, repositionable, flexible J wire. The needle can include centimeter markings for depth placement, and a stabilizer to stabilize the needle. The needle can be a 20 gauge needle with a length ranging from 5 cm to 12.5 cm.

Localization Needles

The coating can be applied to a localization needle (e.g., for use in conjunction with mammography). The localization needle can be, for example, a Hawkins III needle, a “D” wire breast localization needle, an ACCURA™ breast localization needle, or an ACCURA™ II breast localization needle from InterV.

A Hawkins III needle can be a traditional “hardwire” needle, or a “cable,” i.e., a strand of smaller wires, which can provide greater flexibility. The needle can include markings for depth placement, and an end hole for dye injection or fluid aspiration. The needle can be a 20 gauge needle with a length ranging from 3 cm to 12.5 cm.

A “D” wire breast localization needle can have a “D”-shaped cross-section. The needle can include markings for depth placement, a skin retention clip to stabilize the needle, and an end hole for dye injection or fluid aspiration. The needle can be a 20 gauge needle with a length ranging from 3 cm to 15 cm.

An ACCURA breast localization needle can have a springhook hardwire with stiffener design. The needle can include markings for depth placement, a skin retention clip to stabilize the needle, and an end hole for dye injection or fluid aspiration. The needle can be a 20 gauge or 21 gauge needle with a length ranging from 3 cm to 10 cm.

An ACCURA II breast localization needle can have a springhook hardwire with stiffener design, and can be used with a 23 G stiffening cannula. The needle can include markings for depth placement, a skin retention clip to stabilize the needle, and an end hole for dye injection or fluid aspiration. The needle can be a 20 gauge needle with a length ranging from 5 cm to 12.5 cm.

Special Radiology

The coating can be applied to a surface of devices used in special radiology applications. For example, the coating can be applied to devices used in galactography/sialography, to enteroclysis and duodenography catheters, to devices in a TearLeader® stent set, to an HSG catheter, to devices in a fallopian tube set or a cholangiography set, or a Quadra-Fuse multi-pronged injection needle.

Galactography/sialography devices can include a polyethylene catheter or fine blunt cannula, which can be straight, curved, or bent; and a dilator.

Enteroclysis and duodenography catheters can be made of polyvinyl chloride and include an inflatable antireflux balloon. The catheters can have a soft, round tip for atraumatic introduction.

A TearLeader® stent set is used for placing a stent in the nasolacrimal duct. It can have an “S” shape for single step placement. The set can include a 3 F×10 cm dacryocystography catheter with ball tip stylet, a 0.018″ nitinol guidewire, and a 6 F×4.5 cm S-shaped stent with side holes.

A HSG catheter can be used, for example, for radiological hysterosalpingography or hydro hysterosonography. The catheter can include a soft, non-latex balloon which prevents leakage of saline or contrast media. The catheter can have dimensions of, for example, 5 F×40 cm or 7 F×40 cm.

Devices in a fallopian tube set (e.g., for use in salpingography and fallopian tube procedures) can include a 10 F balloon catheter, a 5 F selective salpingography catheter, and/or (for uterine corunal access) a 3 F radiopaque catheter and a nitinol 0.018″ guidewire.

A cholinangiography set can include an 18 G×6.5 cm blunt curved needle with 25 cm connecting tube, and can be used for contrast injection while reducing the risk of puncture of the common bile duct or choledochus.

Vascular Access

The coating can be applied to a device used for vascular access, such as, for example, a guidewire introducer needle, or a vascular dilator. A guidewire introducer needle can be used in an anterior, single-wall arterial/Seldinger percutaneous procedure. The needle can have a non-coring “B” arterial bevel, and an optional winged base plate. The needle can be an 18 G×7 cm needle (for 0.038″ guidewires) or a 19 G×7 cm needle (for 0.035″ guidewires). The needle can be a modified Potts/Cournand needle (e.g., for carotid angiography, direct arterial pressure monitoring, blood sampling, or percutaneous catheterization).

A vascular dilator can be a radiopaque polyethylene dilator, with a smooth rounded tip, and a Luer lock hub for contrast injection and/or guidewire exchange. The dilator can have dimensions of 4 F to 8 F and a length of 20 cm. It can accommodate a 0.035″ or 0.038″ guidewire.

Urology Devices

The coating can be applied to devices used in urological procedures, such as, for example, a ureteral pigtail stent set, catheters, guidewires, or a TRU-CORE I Uro biopsy needle/instrument.

A ureteral pigtail stent set can include an introducer (with a transparent inner catheter and a positioning catheter), a torque handle for manipulating a guidewire, a guidewire (such as a PTFE coated WORKER® guidewire), and the ureteral stent. The stent can be a soft polyurethane with a size of 6 F to 8 F and a length of 24 cm to 28 cm.

Urological accessories, such as catheters and guidewires, can be coated. The catheter can be a pyelography catheter (e.g., with end hole design and centimeter markings); the guidewire can be, for example, a WORKER® or a SURFER® guidewire.

A TRU-CORE I Uro biopsy needle can be an 18 G needle with a 19 mm sample notch, with centimeter markings and an echogenic tip.

Oncology Devices

The coating can be applied to devices used in oncology procedures, such as, for example, a bone marrow biopsy needle, a bone marrow aspiration needle, a bone morrow access needle, a PSS prostate seeding set, or a prostate stabilization set. The bone marrow biopsy needle can be, e.g., a SNARE-LOK needle, a TRAP-LOK needle, or a T-LOK needle.

A PSS prostate seeding needle can be a disposable needle used to facilitate transperineal, radioactive seed implant procedures. The needle can have an echogenic tip for visualization under ultrasound guidance. The needle can have centimeter and half-centimeter markings. A prostate stabilization set can include a needle with a side barb to immobilize the prostate gland during transperineal seeding procedures. The needle can have an echogenic tip.

Stenting

The coating can be applied to a stent or devices used in stenting, such as, for example, a pigtail biliary stent, a guidewire and guiding/pusher catheters for biliary stenting, or a stent introducer system and sizeguide catheter.

A pigtail biliary stent can have a pigtail at each end, each having five sideholes, and can be made of radiopaque polyethylene. It can have dimensions of 7 F to 10 F, with a total length ranging from 6.5 cm to 17.5 cm. The stent can be placed using a guidewire (e.g., a WORKER® guidewire or a hydrophilic coated stainless steel guidewire) and an introducer system (e.g., a 7 F or 10 F biliary stent introducer system).

A guiding/pusher catheter can be, e.g., a 5 F or 6 F PTFE catheter, optionally with a radiopaque band (e.g., of gold); or can be a radiopaque FEP catheter, with a size ranging from 7 F to 11.4 F.

A stent introducer system includes an inner and outer catheter. The inner catheter is made of radiopaque PTFE and the outer catheter is made of radiopaque FEP. The inner catheter has three markers for enhanced visualisation. The inner catheter can have a size of 5 F or 6 F, and can be paired with an outer catheter of size 8.5 F or 10 F, respectively.

A sizeguide catheter can be made of radiopaque PTFE and have 3 distance markers, which are used as a reference point for the radiographic magnification, thus facilitating measurement of the stricture to select the proper stent size. The catheter can have a removable hub for contrast injection, and can also be used as a guiding catheter.

Stone Removal

The coating can be applied to a device for stone removal, e.g., removal of stones from the common bile duct, such as a catheter. The stone removal catheter can be tapered, e.g., having a diameter of 5 F at the distal end and 7 F at the proximal end. The distal end can include an inflatable balloon, with a radiopaque band under the balloon for visualization. The catheter can have dimensions of 7 F (tapered to 5 F at the distal end)×200 cm, and accept a 0.035″ guidewire. An exemplary stone removal catheter is the EXPEL catheter from PBN Medicals.

Colon Decompression

The coating can be applied to a colon decompression catheter, e.g., for colonscopic decompression in toxic megacolon, pseudo obstruction and decompression of the colon proximal to a stricture. The catheter can be made of radiopaque polyvinylchloride, have dimensions of 16 F or 18 F×175 cm, have side holes (e.g., six side holes). The catheter can be used with a guidewire, such as a 0.035″ PTFE coated guidewire.

The foregoing medical equipment can be formed of various materials, including organic and inorganic polymers as well as metal, ceramic, or glass. Organic polymers include polymers or copolymers of, for example, polyurethanes, silicones, polyvinylchloride, polyolefins (including high density and low density polyethylene, and polypropylene), polyamides, latex and metals including steel; as well inorganic polymers. The coatings can be applied to the various materials, as required by the construction of the device being coated.

### EXAMPLES

The examples of coating solutions listed below are illustrative and are not intended to limit the scope of the invention. These compositions are adapted to be used as coatings for mesh, wiry, flat and/or sharp metal surfaces as one or more layers.

Composition 1—5.4 gm low viscosity ½ second nitrocellulose, 2.0 gm dibutylphthalate, 1.5 gm camphor, 1.9 gm polyvinylbutyral in a solvent mixture of 36.0 ml toluene, 13.1 ml butylacetate, 5.9 ml isopropanol, 25.4 ml ethylacetate and 18.1 ml ethanol.

Composition 2—6.6 gm polyvinylpyrrolidone, 63.8 ml denatured ethanol, 23.6 ml ethyl acetate and 12.6 ml dimethylformamide.

Composition 3—1.9 gm ½ second nitrocellulose together with a hydrophilic polymer, 1.5 gm polyvinylpyrrolidone, in 60 ml ethylacetate, 34.4 ml denatured ethanol, 4.6 ml acetic acid and 1 ml isopropanol.

Composition 4—7.5 gm polyvinylpyrrolidone together with a stabilizing polymer, 0.3 gm nitrocellulose, in 73 ml denatured ethanol, 26.8 ml ethylacetate and 0.2 ml isopropanol.

Composition 5—5.9 gm ½ sec. cellulose acetate butyrate and 5.9 gm polyvinylpyrrolidone in 33 ml ethyl acetate and 67 ml chloroform.

Composition 6—5 gm polyvinylpyrrolidone in 95 ml denatured ethanol.

Composition 7—1.9 gm nitrocellulose, 1.1 gm polyester resin, 1.0 gm monobutylester of polymethylvinylether/maleic anhydride copolymer, 0.8 ml isopropanol, 57.8 ml ethylacetate, 33.4 ml denatured ethanol and 8 ml dimethylformamide.

Composition 8—6.6 gm polyvinylpyrrolidone, 63.8 ml denatured ethanol, 23.6 ml ethylacetate and 12.6 ml dimethylformamide.

Additional exemplary compositions, which may be applied in one or more layers:

| | | |
|---|---|---|
| nitrocellulose | 56 | gm |
| camphor | 15 | gm |
| dibutylphthalate | 20 | gm |
| isopropanol | 23 | ml |
| toluene | 225 | ml |
| ethyl acetate | 330 | ml |
| butyl acetate | 96 | ml |
| acetone | 7 | ml |

| | | |
|---|---|---|
| polyvinylpyrrolidone (PVP) | 3 | gm |
| denatured ethanol | 27 | ml |
| ethyl acetate | 10 | ml |
| dimethylformamide | 12 | ml |

| | | |
|---|---|---|
| nitrocellulose | 65 | gm |
| dibutylphthalate | 24 | gm |
| camphor | 18 | gm |
| polyvinylbutyral | 23 | gm |
| acetone | 28 | ml |
| ethanol | 306 | ml |
| butyl acetate | 257 | ml |
| ethyl acetate | 500 | ml |
| toluene | 550 | ml |
| isopropanol | 28 | ml |
| dimethylformamide | 200 | ml |

| | | |
|---|---|---|
| polyvinylpyrrolidone | 1 | gm |
| ethanol | 9 | ml |
| dimethylformamide | 3 | ml |
| water | 0.5 | ml |

| | |
|---|---|
| cellulose acetate propionate | 12.9 gm |
| dibutylphthalate | 4.8 gm |
| camphor | 3.6 gm |
| acetone | 3.2 gm |
| ethyl acetate | 55.7 gm |
| toluene | 58.6 gm |
| butyl acetate | 28.5 gm |
| isopropanol | 5.6 gm |

| | | |
|---|---|---|
| acetonitrile | 5 | ml |
| ethanol | 4.5 | ml |
| PVP (360,000 mw) | 0.5 | gm |

| | | |
|---|---|---|
| cellulose acetate | 12.9 | gm |
| dibutylphthalate | 4.8 | gm |
| camphor | 3.6 | gm |
| methylethylketone | 148.3 | ml |
| dimethylformamide | 20.0 | ml |

| | | |
|---|---|---|
| PVP (360,000 mw) | 0.5 | gm |
| cellulose acetate | 0.1 | gm |
| acetone | 6 | ml |
| ethanol | 4.5 | ml |
| acetic acid | 1.0 | ml |
| methylethylketone | 0.9 | gm |

| | | |
|---|---|---|
| cellulose acetate butyrate | 6.5 | gm |
| polyester resin | 6.0 | gm |
| dibutylphthalate | 2.4 | gm |
| camphor | 1.8 | gm |
| acetone | 2.5 | ml |
| ethyl acetate | 43.6 | ml |
| toluene | 43.6 | ml |
| butylacetate | 22.4 | ml |

| | | |
|---|---|---|
| acetonitrile | 5 | ml |
| ethanol | 4.5 | ml |
| PVP (360,000 mw) | 0.5 | gm |

| | | |
|---|---|---|
| nylon resin | 2 | gm |
| trifluoroethanol | 18 | ml |

| | | |
|---|---|---|
| PVP (360,000 mw) | 1.0 | gm |
| nylon resin | 0.3 | gm |
| ethanol | 9.0 | ml |
| dimethylformamide | 3.0 | ml |
| trifluoroethanol | 2.7 | ml |

| | | |
|---|---|---|
| nitrocellulose | 64.6 | gm |
| dibutylphthalate | 24.3 | gm |
| camphor | 17.9 | gm |
| polyvinylbutyral | 22.5 | gm |
| acetone | 28.4 | ml |
| ethanol | 306.1 | ml |
| butylacetate | 257.0 | ml |
| ethylacetate | 499.2 | ml |
| toluene | 552.8 | ml |
| isopropanol | 27.5 | ml |
| dimethylformamide | 200.0 | ml |

| | | |
|---|---|---|
| PVP | 1.0 | gm |
| ethanol | 9.0 | ml |
| dimethylformamide | 2.0 | ml |

| | | |
|---|---|---|
| nitrocellulose | 32.3 | gm |
| polyurethane | 11.2 | gm |
| dibutylphthalate | 12.2 | gm |
| camphor | 9.0 | gm |
| polyvinylbutyral | 11.2 | gm |
| acetone | 25 | ml |
| ethanol | 254 | ml |
| butylacetate | 225.3 | ml |
| ethylacetate | 439.2 | ml |
| toluene | 467.8 | ml |
| isopropanol | 13.8 | ml |
| dimethylformamide | 100 | ml |

| | | |
|---|---|---|
| PVP | 1.0 | gm |
| nitrocellulose | 0.12 | gm |
| ethanol | 9.0 | ml |
| dimethylformamide | 3.0 | ml |
| ethylacetate | 0.4 | ml |

| | | |
|---|---|---|
| nitrocellulose | 64.6 | gm |
| dibutylphthalate | 24.3 | gm |
| camphor | 17.9 | gm |
| polyvinylbutyral | 22.5 | gm |
| acetone | 28.4 | ml |
| ethanol | 306.1 | ml |
| butylacetate | 257.0 | ml |
| ethylacetate | 449.2 | ml |
| toluene | 552.8 | ml |
| isopropanol | 27.5 | ml |
| dimethylformamide | 200.0 | ml |

| | | |
|---|---|---|
| PVP | 1.0 | gm |
| ethanol | 9.0 | ml |
| dimethylformamide | 2.0 | ml |

| | | |
|---|---|---|
| nitrocellulose | 32.3 | gm |
| polyurethane | 10.0 | gm |
| dibutylphthalate | 12.2 | gm |
| camphor | 9.0 | gm |
| polyvinylbutyral | 11.2 | gm |
| acetone | 25 | ml |
| ethanol | 264 | ml |
| butylacetate | 226.3 | ml |
| ethyl acetate | 439.2 | ml |
| toluene | 467.8 | ml |
| isopropanol | 13.8 | ml |
| dimethyformamide | 100 | ml |

| | | |
|---|---|---|
| PVP | 1.0 | gm |
| ethanol | 9.0 | ml |
| dimethylformamide | 3.0 | ml |
| water | 0.5 | ml |

| | | |
|---|---|---|
| nitrocellulose | 64.6 | gm |
| dibutylphthalate | 24.3 | gm |
| camphor | 17.9 | gm |
| polyvinylbutyral | 22.5 | gm |
| acetone | 28.4 | ml |
| ethanol | 306.1 | ml |
| butylacetate | 257.0 | ml |
| ethyl acetate | 499.2 | ml |
| toluene | 552.8 | ml |
| isopropanol | 27.5 | ml |
| dimethylformamide | 200.0 | ml |

| | | |
|---|---|---|
| PVP | 1.0 | gm |
| ethanol | 9.0 | ml |
| dimethylformamide | 1.0 | ml |

| | | |
|---|---|---|
| PVP | 1.0 | gm |
| ethanol | 9.0 | ml |
| acetic acid | 3.0 | ml |

| | | |
|---|---|---|
| PVP | 0.5 | gm |
| nitrocellulose | 0.056 | gm |
| methylethylketone | 13.7 | ml |
| isopropanol | 0.024 | gm |
| acetic acid | 1.0 | ml |

### Example 1

Chiba and Franseen stainless steel biopsy needles (InterV, Gainesville, Fla.) were coated with the following solution and dried in an oven for 10-15 minutes at 75° C. to improve adhesion of subsequent coatings to the metal substrate.

Coating Solution A

| Component | Amount (grams) |
|---|---|
| Tetrahydrofuran | 72.1 |
| Cyclohexanone | 19.3 |
| Ethylene acrylic acid copolymer | 3.6 |
| Aliphatic polyisocyanate | 1.2 |
| Acrylic polymer | 1.1 |
| Xylene | 1.0 |
| Trichloroacetic acid | 0.9 |
| Melamine formaldehyde polymer | 0.4 |
| Ethyl benzene | 0.2 |
| Butyl alcohol | 0.2 |

The needles then were coated with the following solution and dried in an oven for 10-15 minutes at 75° C. to form an intermediate tie layer to enhance adhesion of the echogenic layer to the primer layer.

Coating Solution B

| Component | Amount (grams) |
|---|---|
| Cyclohexane | 20.1 |
| Tetrahydrofuran | 17.6 |
| Ethyl acetate | 13.5 |
| Benzyl alcohol | 11.0 |
| Cellulose ester polymer | 9.8 |
| n-Butyl acetate | 6.6 |
| Toluene | 4.4 |
| Acrylic polymer | 3.9 |
| Xylene | 3.6 |
| Dibutylphthalate | 2.6 |
| Aliphatic polyurethane | 2.0 |
| D,L-Camphor | 1.9 |
| Melamine formaldehyde polymer | 1.5 |
| Ethyl benzene | 0.7 |
| Butyl alcohol | 0.6 |
| Hydroxy-4-methoxybenzophenone | 0.1 |
| Trichloroacetic acid | 0.1 |

The needles then were coated with the following coating solution, incubated in a humidity chamber (45-46% RH at ˜21° C.) for 10-20 minutes, and cured at 75° C. for 10-15 minutes to form an echogenic coating on the needles.

Coating Solution C

| Component | Amount (grams) |
|---|---|
| Tetrahydrofuran | 30.9 |
| Dimethylsulfoxide | 25.2 |
| Polyether polyisocyanate | 22.1 |
| Acetone | 20.6 |
| Nonionic surfactant | 1.2 |

The use of a primer and bonding layer can improve adhesion of the echogenic coating to various types of substrates to which single layer coatings typically do not adhere well. Other types of medical devices that may be coated in a similar fashion include those having one or more metal or metal alloy components (e.g., stainless steel, nitinol, titanium and its alloys, tantalum, aluminum, cobalt-chromium alloys, and nickel-silver alloys) or components made from polymers such as polyethylene, polypropylene, fluoropolymers such as poly(tetrafluoroethylene) and fluorinated ethylene propylene, polyvinyl chlorides, polyethylene terephthalate, polymethyl methacrylate, and silicone. Specific examples include, but are not limited to needles (e.g., biopsy needles (e.g., MAXICELL needles from Inter V, Gainesville, Fla. or VENA-STICK needles from Inter V, Gainesville, Fla.) aspiration needles, bone marrow biopsy needles, breast localization needles, injection needles, biopsy co-axial introducer needles, bone biopsy needles, guidewire introducer needles, epidural needles, and Huber needles), vascular dilators, guide wires, stents, biopsy site markers (e.g., V-MARK breast biopsy site marker from Inter V, Gainesville, Fla.), retrieval snares, trocars (e.g., feeding trocars, drainage trocars, etc), hemostasis valves, and brachytherapy seeds. Representative examples of devices made from polymeric materials include catheters, and brachytherapy seeds.

### Example 2

Stainless steel wires were coated in Coating Solution A and dried in an oven for 10-15 minutes at 75° C. to improve adhesion of subsequent coatings to the metal substrate.

The wires then were coated in Coating Solution B and dried in an oven for 12-15 minutes at 75° C. to form an intermediate tie layer to enhance adhesion of the echogenic layer to the primer layer.

The wires then were coated in Coating Solution C, incubated in a humidity chamber (70% RH at 40° C.) for 60 minutes, and cured at 75° C. for at least 15 minutes to form an echogenic coating on the wires.

### Example 3

A venous access needle (V-stick by InterV) were coated in Coating Solution A and dried in an oven for 10-15 minutes at 75° C. to improve adhesion of subsequent coatings to the metal substrate.

The needles then were coated in Coating Solution B and dried in an oven for 12-15 minutes at 75° C. to form an intermediate tie layer to enhance adhesion of the echogenic layer to the primer layer.

The needles then were coated in Coating Solution C, incubated in a humidity chamber (70% RH at 40° C.) for 60 minutes, and cured at 75° C. for at least 15 minutes to form an echogenic coating on the needles.

The needles were then sprayed over the length of the coating with an aminofunctional dimethylsiloxane copolymer (MDX4-4159 From Dow Corning) to impart lubricity to the echogenic coating. Subsequent testing indicated a reduction in the penetration force and drag normally associated with the coating, which still maintained its echogenicity.

### Example 4

A venous access needle (V-stick by InterV) were coated in Coating Solution A and dried in an oven for 10-15 minutes at 75° C. to improve adhesion of subsequent coatings to the metal substrate.

The needles then were coated in Coating Solution B and dried in an oven for 12-15 minutes at 75° C. to form an intermediate tie layer to enhance adhesion of the echogenic layer to the primer layer.

The needles then were coated in Coating Solution C, incubated in a humidity chamber (70% RH at 40° C.) for 60 minutes, and cured at 75° C. for at least 15 minutes to form an echogenic coating on the needles.

The needles were then sprayed at the needle tip only with an aminofunctional dimethylsiloxane copolymer (MDX4-4159 From Dow Corning) to reduce the penetration force of the needle upon insertion. Subsequent testing indicated a reduction in the penetration force of the needle while having no deleterious effect on the echogenicity associated with the coating.

### Example 5

Stainless steel wires were coated in Coating Solution A and dried in an oven for 10-15 minutes at 75° C. to improve adhesion of subsequent coatings to the metal substrate.

The wires then were coated in Coating Solution B and dried in an oven for 12-15 minutes at 75° C. to form an intermediate tie layer to enhance adhesion of the echogenic layer to the primer layer.

The wires then were coated in Coating Solution D, incubated in a humidity chamber (70% RH at 40° C.) for 60 minutes, and cured at 75° C. for at least 15 minutes to form an echogenic coating on the wires.

Coating Solution D

| Component | Amount (grams) |
|---|---|
| Tetrahydrofuran | 29.97 |
| Dimethylsulfoxide | 24.44 |
| Polyether polyisocyanate | 21.44 |
| Acetone | 19.98 |
| Pyridine | 3.0 |
| Nonionic surfactant | 1.16 |

### Example 6

The stainless steel trocar of a drainage catheter (SKATER® Nephrostomy catheter from PBN Medicals/InterV, Denmark) was coated in Coating Solution A and dried in an oven at 75° C. for 15 minutes to improve adhesion of subsequent coatings to the metal substrate.

The trocar was coated with Coating Solution B and dried at 75° C. for 15 minutes to form an intermediate tie layer to enhance adhesion of the echogenic layer to the primer layer.

The trocar then was coated with Coating Solution C, incubated in a humidity chamber (about 45-46% RH) for 20 minutes, and then dried at 75° C. for 15 minutes to form an echogenic coating on the trocar.

### Example 7

A tip of a polyurethane catheter (SKATER Nephrostomy catheter from PBN Medicals/InterV, Denmark) was coated in Coating Solution C, incubated in a humidity chamber (about 45-46% RH) for 20 minutes and dried at 75° C. for 15 minutes to form an echogenic coating on the catheter tip.

Other examples of medical devices which may coated directly with echogenic coatings as described herein include devices having polymeric components made of, for example, polyurethane, such as catheters (drainage catheters, catheters for use with retrieval snares, dilators, ureteral catheters, guiding and vessel sizing catheters, nephrostomy catheters, nasal duct drainage catheters, colon decompression catheters, stone removal catheters).

### Example 8

The tip of a polyurethane nephrostomy catheter (SKATER from PBN Medicals/InterV, Denmark) was coated in Coating Solution B and dried at 75° C. for 15 minutes to form a tie layer to enhance adhesion of the echogenic layer to the substrate.

The catheter then was coated with Coating Solution C, treated in a humidity chamber (about 45-50% RH) for 20 min and then dried at 75° C. for 15 minutes to form an echogenic coating.

Other examples of medical devices which may be coated with a similar two-layer coating system include polyurethanes, polyamides, and silicones such as catheters, sutures, and angioplasty balloons.

### Example 9

A length of a polyurethane drainage catheter (SKATER Centesis catheter from PBN Medicals/InterV, Denmark) was coated in Coating Solution C, incubated in a humidity chamber (46% RH) for 12 minutes and dried at 75° C. for 12 minutes to form an echogenic coating on the catheter.

### Example 10

The length of a polyurethane drainage catheter (SKATER Centesis catheter from PBN Medicals/InterV, Denmark) was coated in Coating Solution B and dried at 75° C. for 12 minutes to form a tie layer to enhance adhesion of the echogenic layer to the substrate.

The catheter then was coated with Coating Solution C, treated in a humidity chamber (46% RH) for 12 min and then dried at 75° C. for 12 minutes to form an echogenic coating.

### Example 11

A fluoroethylene propylene (FEP) vascular sheath was coated with Coating Solution C, treated in a humidity chamber (about 45-50% RH) for 20 minutes, and then dried at 75° C. for 15 minutes to form a coating which generated contrast under ultrasound. Other examples of medical devices which may coated directly with echogenic coatings as described herein include devices having polymeric components made of polyurethane, polyethylene, and polypropylene such as catheters (drainage catheters, catheters for use with retrieval snares, dilators, ureteral catheters, guiding and vessel sizing catheters), sutures, angioplasty balloons, and stents.

### Example 12

A fluoroethylene propylene (FEP) vascular sheath was plasma treated with Argon gas. The sheath was immediately coated with Coating Solution B and dried in an oven at 75° C. for 15 minutes.

The sheath was then coated with Coating Solution C, treated in a humidity chamber (about 46% RH) for 20 minutes, and dried at 75° C. for 20 minutes to form a coating which generated contrast under ultrasound.

### Example 13

The nitinol snare portion of a retrieval snare device (e.g., ENSNARE from Inter V, Gainesville, Fla.) is coated in Coating Solution A and dried in an oven for 12-15 minutes at 75° C. to improve adhesion of subsequent coatings to the metal substrate.

The snares are then coated with Coating Solution B and dried in an oven for 12-15 minutes at 75° C. to form an intermediate tie layer to enhance adhesion of the echogenic layer to the primer layer.

The snares are then coated with Coating Solution C, incubated in a humidity chamber (45-46% RH) for 20 minutes, and dried at 75° C. for 15 minutes to form an echogenic coating on the snare.

### Example 14

A sample of surgical tape is coated with an echogenic coating to facilitate ultrasonic visualization of a surgical instrument (e.g., scalpel, scissors, forceps, clamps, retractors, etc) during or after a surgical procedure. After a surgical procedure, a patient is exposed to ultrasound to determine whether the surgical instrument has been left in the body and the instrument may be removed prior to closure.

A sample of surgical tape may be coated via spray coating. Initial primer layers, may or may not be necessary, but a coating solution similar to Coating Solution C would be the final layer used and would be applied to only one side of the surgical tape.

### Example 15

The device of choice (stainless steel or polymer substrate) is coated with Coating Solution A and dried in an oven for 12-15 minutes at 75° C. to improve adhesion of subsequent coatings to the metal substrate.

The device is then coated with Coating Solution B and dried in an oven for 12-15 minutes at 75° C. to form an intermediate tie layer to enhance adhesion of the echogenic layer to the primer layer.

The device is then coated with Coating Solution C, incubated in a humidity chamber (45-46% RH) for at least 10 minutes, and dried at 75° C. for at least 10 minutes to form an echogenic coating on the device.

The device is then coated with Coating Solution E and then dried at 75° C. for at least 15 minutes to form a lubricious coating over the echogenic layer.

Coating Solution E

| Component | Amount (grams) |
|---|---|
| Ethanol | 37.50 |
| Benzyl alcohol | 34.80 |
| Isopropanol | 17.40 |
| Cyclohexanone | 2.70 |
| Polyvinylpyrrolidone (PVP K90) | 5.80 |
| Nitrocellulose, ¼″ RS | 0.0009 |
| 4-Butyrolactone | 0.0091 |
| Polyethylene glycol (MW400) | 1.80 |

### Example 16

A stainless steel substrate (wire or needle) was coated with Coating Solution A and dried in an oven for 15 minutes at 75° C. to improve adhesion of subsequent coatings to the metal substrate.

The samples then were coated with Coating Solution B doped with polymeric gas/liquid containing microparticles (1% weight/weight) (EXPANCEL microparticles from Akzo Nobel, Expancel Inc. US, Duluth, Ga.) and dried in an oven for 15 minutes at 75° C. to form an echogenic layer on the device.

The samples then were coated with Coating Solution F and dried in an oven for at least 30 minutes at 75° C.

Coating Solution F

| Component | Amount (grams) |
|---|---|
| Chronoflex AR | 13.29 |
| Dimethylacetamide | 7.87 |
| Anisole | 26.50 |
| Methylethylketone | 28.75 |
| n-Butanol | 19.17 |
| H-15 Nitrocellulose | 4.42 |

A smooth echogenic coating was produced that adhered well to the substrate and resisted abrasion under wet and dry conditions. The EXPANCEL microparticles were incorporated at weights of 0.06%-10% and maintained echogenicity across this range. Incorporation of echogenic microparticles into an intermediate coating layer may produce a medical device which can remain visible under ultrasound in vivo for an extended period of time (e.g., weeks or months).

Two biocompatibility tests were performed, hemolysis and MEM elution cytotoxicity. There was 0% average hemolysis and no cytotoxic reaction for these tests.

Other types of medical devices that may be coated in a similar fashion include those having one or more metal or metal alloy components (e.g., stainless steel, nitinol, titanium and its alloys, tantalum, aluminum, cobalt-chromium alloys, and nickel-silver alloys) or components made from polymers such as polyethylene, polypropylene, fluoropolymers such as poly(tetrafluoroethylene) and fluorinated ethylene propylene, polyvinyl chlorides, polyethylene terephthalate, polymethyl methacrylate, and silicone. Specific examples include, but are not limited to needles (e.g. biopsy needles (e.g., MAXICELL needles from Inter V, Gainesville, Fla. or VENA-STICK needles from Inter V, Gainesville, Fla.) aspiration needles, bone marrow biopsy needles, breast localization needles, injection needles, biopsy co-axial introducer needles, bone biopsy needles, guidewire introducer needles, epidural needles, and Huber needles), vascular dilators, guide wires, stents, biopsy site markers (e.g., V-MARK breast biopsy site marker from Inter V, Gainesville, Fla.), retrieval snares, trocars (e.g., feeding trocars, drainage trocars, etc), hemostasis valves, and brachytherapy seeds. Representative examples of devices made from polymeric materials include catheters, stents, and brachytherapy seeds.

### Example 17

A stainless steel substrate (wire or needle) was coated with Coating Solution A and dried in an oven for 15 minutes at 75° C. to improve adhesion of subsequent coatings to the metal substrate.

The samples then were coated with Coating Solution B doped with polymeric gas/liquid containing microparticles (1% weight/weight) (EXPANCEL microparticles) and dried in an oven for 15 minutes at 75° C. to form an echogenic layer on the device.

The samples then were dip coated with the hydrophilic Coating Solution E and dried in an oven for at least 30 minutes at 75° C.

A smooth echogenic coating was produced that adhered well to the substrate, resisted abrasion under wet and dry conditions, and was lubricious when wet.

Incorporation of microparticles into an intermediate coating layer can produce a medical device which may remain visible under ultrasound in vivo for an extended period of time (e.g., weeks or months). Two biocompatibility tests were performed, hemolysis and MEM elution cytotoxicity. There was 0% average hemolysis and a slight cytotoxic reaction observed for these tests.

Other types of medical devices that may be coated in a similar fashion include those having one or more metal or metal alloy components (e.g., stainless steel, nitinol, titanium and its alloys, tantalum, aluminum, cobalt-chromium alloys, and nickel-silver alloys) or components made from polymers such as polyethylene, polypropylene, fluoropolymers such as poly(tetrafluoroethylene) and fluorinated ethylene propylene, polyvinyl chlorides, polyethylene terephthalate, polymethyl methacrylate, and silicone. Specific examples include, but are not limited to needles (e.g., biopsy needles (e.g., MAXICELL needles from Inter V, Gainesville, Fla. or VENA-STICK needles from Inter V, Gainesville, Fla.) aspiration needles, bone marrow biopsy needles, breast localization needles, injection needles, biopsy co-axial introducer needles, bone biopsy needles, guidewire introducer needles, epidural needles, and Huber needles), vascular dilators, guide wires, stents, biopsy site markers (e.g., V-MARK breast biopsy site marker from Inter V, Gainesville, Fla.), retrieval snares, trocars (e.g., feeding trocars, drainage trocars, etc), hemostasis valves, and brachytherapy seeds. Representative examples of devices made from polymeric materials include catheters, stents, and brachytherapy seeds.

### Example 18

A stainless steel substrate (wire or needle) was coated with Coating Solution A doped with polymeric gas/liquid containing microparticles (between 0.5 and 5% weight/weight) (EXPANCEL microparticles) and dried in an oven for 15 minutes at 75° C. to form an echogenic layer on the device.

The sample then was coated with Coating Solution B and dried in an oven for at least 15 minutes at 75° C.

A smooth echogenic coating was produced that adhered well to the substrate and resisted abrasion under wet and dry conditions. Incorporation of echogenic microparticles into an intermediate coating layer may produce a medical device which may remain visible under ultrasound in vivo for an extended period of time (e.g., weeks or months).

### Example 19

A stainless steel substrate (wire or needle) was coated with Coating Solution A doped with polymeric gas/liquid containing microparticles (between 0.5 and 5% weight/weight) (EXPANCEL microparticles) and dried in an oven for 15 minutes at 75° C. to form an echogenic layer on the device.

The sample then was coated with Coating Solution B and dried in an oven for at least 15 minutes at 75° C.

The samples then were dip coated with the hydrophilic Coating Solution E and dried in an oven for at least 30 minutes at 75° C.

A smooth echogenic coating was produced that adhered well to the substrate and resisted abrasion under wet and dry conditions, and was lubricious when wet. Incorporation of echogenic microparticles into an intermediate coating layer may produce a medical device which may remain visible under ultrasound in vivo for an extended period of time (e.g., weeks or months).

### Example 20

A stainless steel substrate (wire or needle) was coated with Coating Solution A and dried in an oven for 15 minutes at 75° C. to improve adhesion of subsequent coatings to the metal substrate.

The sample then was coated with Coating Solution B doped with polymeric gas/liquid containing microparticles (between 0.5% and 10% weight/weight) (EXPANCEL microparticles) and dried in an oven for at least 15 minutes at 75° C. to form an echogenic layer on the device.

A smooth echogenic coating was produced that adhered well to the substrate and resisted abrasion under wet and dry conditions. Incorporation of echogenic microparticles may produce a medical device which may remain visible under ultrasound in vivo for an extended period of time (e.g., weeks or months).

### Example 21

A stainless steel substrate (wire or needle) was coated with Coating Solution A and dried in an oven for 15 minutes at 75° C. to improve adhesion of subsequent coatings to the metal substrate.

The sample then was coated with Coating Solution B dried in an oven for at least 15 minutes at 75° C.

The samples then were dip coated with the hydrophilic Coating Solution F doped with polymeric gas/liquid containing microparticles (between 0.5% and 10% weight/weight) (EXPANCEL microparticles) and dried in an oven for at least 30 minutes at 75° C. to form an echogenic layer on the device.

A smooth echogenic coating was produced that adhered well to the substrate and resisted abrasion under wet and dry conditions. Incorporation of echogenic microparticles into an intermediate coating layer can produce a medical device which may remain visible under ultrasound in vivo for an extended period of time (e.g., weeks or months).

### Example 22

A stainless steel substrate (wire or needle) was coated with Coating Solution A and dried in an oven for 15 minutes at 75° C. to improve adhesion of subsequent coatings to the metal substrate.

The sample then was coated with Coating Solution B doped with polymeric gas/liquid containing microparticles (0.5% and 10% weight/weight) (EXPANCEL microparticles) and dried in an oven for at least 15 minutes at 75° C. to form an echogenic layer on the device.

The sample then was coated with Coating Solution B and dried in an oven for 15 minutes at 75° C.

A smooth echogenic coating was produced that adhered well to the substrate and resisted abrasion under wet and dry conditions. Incorporation of echogenic microparticles may produce a medical device which may remain visible under ultrasound in vivo for an extended period of time (e.g., weeks or months).

### Example 23

A stainless steel substrate (wire or needle) was coated with Coating Solution A and dried in an oven for 15 minutes at 75° C. to improve adhesion of subsequent coatings to the metal substrate.

The samples then were coated with Coating Solution B and dried in an oven for 15 minutes at 75° C.

The samples then were dip coated with Coating Solution F doped with polymeric gas/liquid containing microparticles (between 0.5% and 10% weight/weight) (EXPANCEL microparticles) and dried in an oven for at least 30 minutes at 75° C. to form an echogenic layer on the device.

The samples then were coated with Coating Solution F and dried in an oven for 30 minutes at 75° C.

A smooth echogenic coating was produced that adhered well to the substrate and resisted abrasion under wet and dry conditions. Incorporation of echogenic microparticles may produce a medical device which may remain visible under ultrasound in vivo for an extended period of time (e.g., weeks or months).

### Example 24

A stainless steel substrate (wire or needle) was coated with Coating Solution A doped with polymeric gas/liquid containing microparticles (between 0.5 and 5% weight/weight) (EXPANCEL microparticles) and dried in an oven for 15 minutes at 75° C. to form an echogenic layer on the device.

### Example 25

A stainless steel substrate (wire or needle) was coated with Coating Solution G doped with polymeric gas/liquid containing microparticles (between 1 and 5% weight/weight) (EXPANCEL microparticles) and dried in an oven for 15 minutes at 75° C. to form an echogenic layer on the device.

Coating Solution G

| Component | Concentration |
|---|---|
| Propylene glycol methyl ether acetate | 24.5-26.0% |
| Methyl ethyl ketone | 20.5-22.5% |
| Ethanol | 13.5-14.5% |
| Xylene | 13-14% |
| Vinyl acetate/acrylic copolymer | 5.0-6.0% |
| Butanol | 4.5-5.5% |
| Dipropylene glycol monomethyl ether acetate | 3.5-5.5% |
| Formaldehyde copolymer | 3.5-4.5% |
| Ethyl benzene | 2.5-3.0% |
| Isopropyl alcohol | 1.0-2.0% |
| Methanol | 1.0-2.0% |
| Phenol | <1% |
| Formaldehyde | <0.01% |

Other embodiments are within the scope of the following claims.

What is claimed is:

1. A medical device comprising a surface coated with a first layer proximal to the surface, a second layer coated on the first layer, the second layer including a plurality of microparticles, and a third layer coated on the second layer, at least one layer including an echogenic structure.

2. The device of claim 1, wherein the first layer includes an ethylene acrylic acid copolymer and an isocyanate.

3. The device of claim 1, wherein the second layer includes a plurality of microparticles.

4. The device of claim 3, wherein the plurality of microparticles includes a polymeric gas/liquid containing microparticle.

5. The device of claim 1, wherein the third layer includes a cellulose based polymer.

6. The device of claim 1, wherein the device is a needle, a vascular dilator, a guide wire, a stent, a biopsy site marker, a retrieval snare, a trocar, a hemostasis valve, or a brachytherapy seed.

7. The device of claim 6, wherein the needle is a disposable soft tissue biopsy needle, an aspiration needle, a bone marrow biopsy needle, a breast localization needle, an injection needle, a biopsy co-axial introducer needle, a bone biopsy needle, a guidewire introducer needle, an epidural needle, or a Huber needle.

8. The device of claim 6, wherein the device is a vascular retrieval snare.

9. A medical device comprising a surface coated with a first layer proximal to the surface, the first layer including an ethylene acrylic acid copolymer and an isocyanate; a second layer coated on the first layer, the second layer including a plurality of microparticles; and a third layer coated on the second layer, the third layer including a cellulose based polymer.

10. The device of claim 9, wherein the isocyanate of the first layer is an aromatic isocyanate.

11. The device of claim 9, wherein the first layer further includes an acrylic polymer and a melamine formaldehyde polymer.

12. The device of claim 9, wherein the plurality of microparticles includes a polymeric gas/liquid containing microparticle.

13. The device of claim 9, wherein the third layer further includes a polyurethane.

14. The device of claim 9, wherein the third layer further includes a polyvinylpyrrolidone.

15. The device of claim 9, wherein the device is a needle, a vascular dilator, a guide wire, a stent, a biopsy site marker, a retrieval snare, a trocar, a hemostasis valve, or a brachytherapy seed.

16. The device of claim 15, wherein the needle is a disposable soft tissue biopsy needle, an aspiration needle, a bone marrow biopsy needle, a breast localization needle, an injection needle, a biopsy co-axial introducer needle, a bone biopsy needle, a guidewire introducer needle, an epidural needle, or a Huber needle.

17. The device of claim 15, wherein the device is a vascular retrieval snare.

18. A method of coating a surface of a device, comprising forming a coating on the surface, wherein the coating includes an echogenic structure and a plurality of microparticles.

19. The method of claim 18, wherein forming the coating includes contacting the surface with a first coating solution including a first solvent, an ethylene acrylic acid copolymer and an isocyanate.

20. The method of claim 19, wherein forming the coating includes contacting the surface with a second coating solution including a second solvent and a plurality of microparticles.

21. The method of claim 20, wherein forming the coating includes contacting the surface with a third coating solution including a third solvent and a cellulose based polymer.

22. The method of claim 21, wherein the isocyanate of the first coating solution is an aromatic isocyanate.

23. The method of claim 21, wherein the first coating solution further includes an acrylic polymer and a melamine formaldehyde polymer.

24. The method of claim 21, wherein the plurality of microparticles includes a polymeric gas/liquid containing microparticle.

25. The method of claim 21, wherein the third coating solution further includes a polyurethane.

26. The method of claim 21, wherein the third coating solution further includes a polyvinylpyrrolidone.

27. The method of claim 21, wherein the device is a needle, a vascular dilator, a guide wire, a stent, a biopsy site marker, a retrieval snare, a trocar, a hemostasis valve, or a brachytherapy seed.

28. The method of claim 27, wherein the needle is a disposable soft tissue biopsy needle, an aspiration needle, a bone marrow biopsy needle, a breast localization needle, an injection needle, a biopsy co-axial introducer needle, a bone biopsy needle, a guidewire introducer needle, an epidural needle, or a Huber needle.

29. The method of claim 27, wherein the device is a vascular retrieval snare.

30. A medical device comprising coating on a surface of the device, wherein the coating comprises a plurality of echogenic microparticles.

31. The device of claim 30, wherein the plurality of microparticles include a polymeric microparticle containing a gas or liquid.

32. The device of claim 31, wherein the polymeric microparticle comprises isopentane, isobutane, a fluorocarbon, air, nitrogen, carbon dioxide, argon, helium, or oxygen.

33. The device of claim 30, wherein the coating includes a first layer proximal to the surface of the device.

34. The device of claim 33, wherein the coating further includes a second layer coated on the first layer.

35. The device of claim 34, wherein the coating further includes a third layer coated on the second layer.

36. The device of claim 30, wherein the surface is stainless steel, a stainless steel alloy, nitinol, titanium, a titanium alloy, tantalum, aluminum, a cobalt-chromium alloy, a nickel-silver alloy, polyethylene, polypropylene, a fluoropolymer, a polyvinyl chloride, polyethylene terephthalate, polymethyl methacrylate, and silicone.

37. The device of claim 30, wherein the device is a needle, a vascular dilator, a guide wire, a stent, a biopsy site marker, a retrieval snare, a trocar, a hemostasis valve, catheters, or a brachytherapy seed.

38. The device of claim 37, wherein the catheter is a drainage catheter.

39. The device of claim 37, wherein the device is a vascular access needle.

40. The device of claim 37, wherein the device is a vascular retrieval snare.

41. The device of claim 37, wherein the needle is a soft tissue biopsy needle, an aspiration needle, a bone marrow biopsy needle, a breast localization needle, an injection needle, a biopsy co-axial introducer needle, a bone biopsy needle, a guidewire introducer needle, an epidural needle, or a Huber needle.

42. The device of claim 37, wherein the needle is disposable.

43. The device of claim 33, wherein the first layer comprises an isocyanate.

44. The device of claim 43, wherein the isocyanate is an aromatic isocyanate.

45. The device of claim 33, wherein the first layer comprises an acrylic polymer and a melamine formaldehyde polymer.

46. The device of claim 33, wherein the first layer comprises a vinyl acetate-acrylic copolymer and a formaldehyde copolymer.

47. The device of claim 33, wherein the first layer comprises an ethylene acrylic acid copolymer and an epoxy-bisphenol A copolymer.

48. The device of claim 47, wherein the first layer further comprises a polyurethane.

49. The device of claim 48, wherein the polyurethane is an aliphatic polycarbonate based polyurethane.

50. The device of claim 34, wherein the second layer comprises a cellulose based polymer.

51. The device of claim 34, wherein the second layer further comprises a polyurethane.

52. The device of claim 34, wherein the second layer further comprises a polyvinylpyrrolidone.

53. The device of claim 34, wherein the second layer further comprises a silicon based polymer.

54. The device of claim 35, wherein the third layer comprises a cellulose based polymer.

55. The device of claim 35, wherein the third layer further comprises a polyurethane.

56. The device of claim 35, wherein the third layer further comprises a polyvinylpyrrolidone.

57. The device of claim 35, wherein the third layer further comprises a silicone.

58. The device of claim 34, wherein the microparticles are substantially localized in the second layer.

59. The device of claim 30, wherein the coating is a lubricious coating.

60. The device of claim 34, wherein the first layer includes the microparticles and the second layer is lubricious.

61. A composition comprising: a plurality of echogenic microparticles suspended in a first polymer matrix.

62. The composition of claim 61, wherein the first polymer matrix includes an ethylene acrylic acid copolymer, an isocyanate, a cellulose based polymer, an acrylic polymer, a melamine formaldehyde polymer, a polyurethane, or a polyvinylpyrrolidone.

63. The composition of claim 61, wherein the plurality of microparticles includes a polymeric gas/liquid containing microparticle.

64. The composition of claim 61, further comprising a solvent.

65. The composition of claim 61, wherein the first polymer matrix is coated on a second polymer matrix.
